(19) United States
(12) Patent Application Publication
(10) Pub. No.: US 2023/0313386 A1
(43) Pub. Date: Oct. 5, 2023

(54) METHODS FOR EXTRACTING CO2 FROM METAL CARBONATES AND USE THEREOF

(71) Applicant: Sublime Systems, Inc., Somerville, MA (US)

(72) Inventors: Leah D. ELLIS, Somerville, MA (US); Yet-Ming CHIANG, Weston, MA (US); Miki L. CHIANG, Weston, MA (US)

(73) Assignee: Sublime Systems, Inc., Somerville, MA (US)

(21) Appl. No.: 18/017,221

(22) PCT Filed: Jul. 22, 2021
(86) PCT No.: PCT/US2021/042746

§ 371 Date: Jan. 20, 2023

Related U.S. Application Data
(60) Provisional application No. 63/055,223, filed on Jul. 22, 2020.

(51) Int. Cl. A01G 7/02; C12M 1/107; C12M 1/00; C12N 1/12; C25B 1/01; C25B 9/05; C25B 15/08; C25B 9/17; A23L 17/60; A23K 10/30; A01G 33/00; C12P 7/06

(52) U.S. Cl. CPC C25B 1/01; A01G 7/02; A01G 33/00; A23K 10/30; A23L 17/60; C12M 21/04; C12M 43/02; C12N 1/12; C12P 7/065; C25B 9/05; C25B 9/17; C25B 15/08

(57) ABSTRACT

Various embodiments may include systems, methods, and devices in which acid produced by a reactor, such as an electrochemical reactor or other type acid producing reactor, is used to produce carbon dioxide (CO₂) from a carbonate and the produced CO₂ is used, or made available for use, for one or more purposes. In some embodiments, the electrochemical reactor may be powered by a renewable energy source.

### CROSS-REFERENCE TO RELATED APPLICATIONS

This application is a national stage application under 35 U.S.C. § 371 of International Application No. PCT/US2021/042746, filed internationally on Jul. 22, 2021, which claims priority to U.S. Provisional Application No. 63/055,223, filed on Jul. 22, 2020, the entire contents of each priority application is hereby incorporated by reference.

### BACKGROUND

CO₂ is used in industry for a variety of purposes. Common sources of CO₂ include mining, carbon capture from combustion gases, large-scale fermentation, oil refining activities, and ammonia production from natural gas. In some cases, CO₂ must be purified before use, and purification is energy-intensive, capital-intensive, and costly.

### SUMMARY

The various embodiments provide methods, devices, materials, and systems for making high-purity CO₂ from mineral carbonates.

Various embodiments may include systems, methods, and devices in which acid produced by a reactor, such as an electrochemical reactor or other type reactor, is used to produce carbon dioxide (CO₂) from a carbonate and the produced CO₂ is used, or made available for use, for one or more purposes. In some embodiments, when the reactor is an electrochemical reactor, the electrochemical reactor may be powered by a renewable energy source.

Various embodiments may include method, comprising: using a reactor to produce at least an acid; releasing CO₂ by dissolution of a metal carbonate in the acid; and using the CO₂ to improve a health, a grow rate, and/or a yield of an organism.

Various embodiments may include an apparatus, comprising: a reactor that is configured to produce at least an acid; a subreactor configured for the dissolution of a metal carbonate in the acid; and a means of collecting CO₂ produced upon dissolution of the metal carbonate in the acid.

Various embodiments may include a system, comprising: a renewable electricity source; a reactor; and a facility containing living organisms.

Various embodiments may include a system, comprising: a reactor configured to produce at least an acid; a second device configured to produce CO₂ at least in part from dissolution of a metal carbonate in the acid; and a collection device configured to: collect the produced CO₂ produced upon dissolution of the metal carbonate in the acid; and provide the produced CO₂ to a storage system and/or operating system, wherein the storage system and/or operating system is configured to enable the produced CO₂ to be applied to, or used by, another system.

A method, comprising: electrochemically dissolving a metal carbonate to release CO₂; and using the released CO₂ to improve a health, a growth rate, and/or a yield of an organism.

A system, comprising: an electrochemical device configured to electrochemically dissolve a metal carbonate to release CO₂; and a collection device configured to collect the released CO₂ and provide the collected CO₂ to a storage system and/or operating system, wherein the storage system and/or operating system are configured to enable the collected CO₂ to be applied to, or used by, another system for one or more purposes.

A method, comprising: dissolving a metal carbonate using an acid to release CO₂; and using the released CO₂ to improve a health, a growth rate, and/or a yield of an organism.

Various embodiments, may include a system comprising: an electrochemical reactor configured to produce at least an acid; a second device configured to produce CO₂ at least in part from dissolution of a metal carbonate in said acid; and a collection device configured to collect the produced CO₂ produced upon dissolution of said metal carbonate in said acid and provide the produced CO₂ to a storage system and/or operating system, wherein the storage system and/or operating system are configured to enable the produced CO₂ to be applied to, or used by, another system for one or more purposes. In some embodiments, the system may further comprise a renewable power source providing power to the electrochemical reactor. In some embodiments, the second device is included in the electrochemical reactor or is separate from the electrochemical reactor. In some embodiments, the reactor is a subreactor. In some embodiments, the metal carbonate is part of a material containing metal carbonate. In some embodiments, the material containing metal carbonate is a natural material, synthesized material, or waste material. In some embodiments, the one or more purposes comprise agricultural or aquaculture purposes. In some embodiments, the one or more purposes comprise use as an inert gas for chemical processes, welding, as a lasing medium, preventing spoilage of foods and other air-sensitive materials, to extinguish fires, to dilute flammable or toxic vapours, as a non-reactive cooling gas; as a toxic gas to terminate or subdue animals, as a medical gas, as a propellant, as a food additive, as a reagent, as a component for the production of building materials, as a pest control mechanism, as an algae growth promotor, as a coral growth promotor, as an oil recovery pressurizing and/or flow agent, as a cleaning agent, as a solvent, or as a refrigerant.

### BRIEF DESCRIPTION OF THE FIGURES

The accompanying drawings, which are incorporated herein and constitute part of this specification, illustrate example embodiments of the claims, and together with the general description given above and the detailed description given below, serve to explain the features of the claims.

FIG. 1A illustrates an example system according to various embodiments.

FIG. 1B illustrates another example system according to various embodiments.

FIG. 2 illustrates a specific example system that generates CO₂ according to various embodiments.

FIG. 3 illustrates a reactor according to various embodiments comprising a first electrode and a second electrode.

### DETAILED DESCRIPTION

The following examples are provided to illustrate various embodiments of the present systems and methods of the present inventions. These examples are for illustrative purposes, may be prophetic, and should not be viewed as limiting, and do not otherwise limit the scope of the present inventions.

The various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the claims. The following description of the embodiments of the invention is not intended to limit the invention to these embodiments but rather to enable a person skilled in the art to make and use this invention. Unless otherwise noted, the accompanying drawings are not drawn to scale.

As used herein, unless stated otherwise, room temperature is 25° C. And, standard temperature and pressure is 25° C. and 1 atmosphere. Unless expressly stated otherwise all tests, test results, physical properties, and values that are temperature dependent, pressure dependent, or both, are provided at standard ambient temperature and pressure.

Generally, the terms “about” or “near” and the symbol “~” as used herein unless specified otherwise is meant to encompass a variance or range of ±10%, the experimental or instrument error associated with obtaining the stated value, and preferably the larger of these.

As used herein unless specified otherwise, the recitation of ranges of values herein is merely intended to serve as a shorthand method of referring individually to each separate value falling within the range. Unless otherwise indicated herein, each individual value within a range is incorporated into the specification as if it were individually recited herein.

Various embodiments include devices, methods, and systems an acid is produced that is subsequently reacted with a carbonate, releasing carbon dioxide (CO₂) in a form useful for industrial and agricultural purposes as described herein. In various embodiments, the acid is produced by a reactor. In some embodiments, the reactor may be a chemical reactor, such as a batch reactor, stirred-tank reactor, etc. In some embodiments, the reactor may be an electrochemical reactor, such as an electrolyzer, a chlor-alkali reactor, an electrodialysis unit, etc.

Various embodiments include devices, methods, and systems wherein electricity is used to produce at least an acid that is subsequently reacted with a carbonate, releasing carbon dioxide (CO₂) in a form useful for industrial and agricultural purposes as described herein.

A general method according to various embodiments uses electrochemistry to produce acid that is reacted with natural minerals or synthesized or waste materials containing metal carbonates (e.g., limestone or dolomite) to release bound CO₂. In certain embodiments, the electrolytic reactor may be an electrolyzer, a chlor-alkali reactor, an electrodialysis unit, or other such electrochemical reactor that produces at least an acid. In some embodiments, the acid is produced as an aqueous solution by the reactor and collected for use in dissolving components of said metal carbonate. In some embodiments, the electrochemical reactor simultaneously produces an acid and a base. An example of such a reactor is a neutral-water electrolyzer. In other embodiments, the electrochemical reactor produces a base and a compound that can subsequently be converted to an acid. An example of such a reactor is a chlor-alkali reactor, which produces NaOH base concurrently with producing chlorine gas. The chlorine gas may be subsequently reacted with water in an “acid burner” to produce hydrochloric acid. In some embodiments, the system includes a vessel in which the metal carbonate is reacted with acid having a pH low enough to cause the evolution of gaseous CO₂. In some embodiments, the system includes apparatus to remove moisture or humidity from the CO₂, including for example compressors, condensers (cold traps) or water-absorbing media. In some embodiments, the system includes apparatus to compress the gaseous CO₂ into a liquid, a supercritical fluid or a gas. In some embodiments, the system includes a pressure vessel configured such that the gaseous CO₂ is generated under pressure, such as a pressure above one atmosphere, above two atmospheres, above three atmospheres, greater than three atmospheres, etc. In various embodiments, generating the CO₂ under pressure may be beneficial as less energy may be needed to pressurize the produced CO₂ for storage and/or use as the produced CO₂ is already under pressure. In some embodiments, the mineral-rich solution resulting from the reaction of acid with the mineral carbonate is reacted with the alkaline solution produced by the electrochemical reactor. In some embodiments, this causes the precipitation of metal hydroxides that can be collected and used for various purposes. In some embodiments, the neutral-pH solution resulting from the neutralization of acidic and basic streams is purified and returned to the electrolyzer, where it is used as an electrolyte, and in which it is converted again into acid and base. In some embodiments, the electrolyzer is powered by renewable electricity.

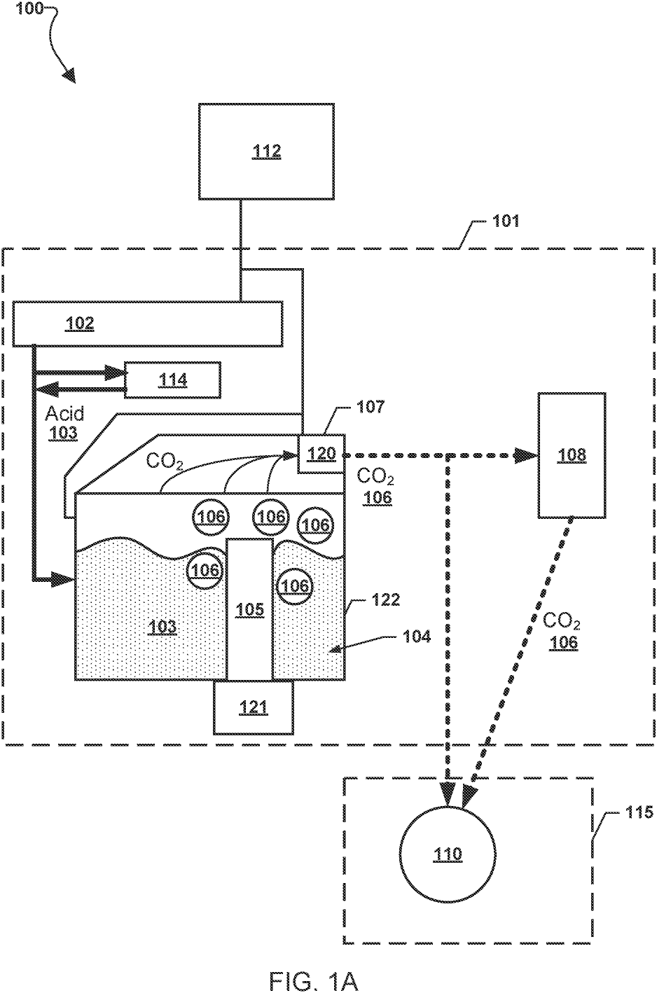

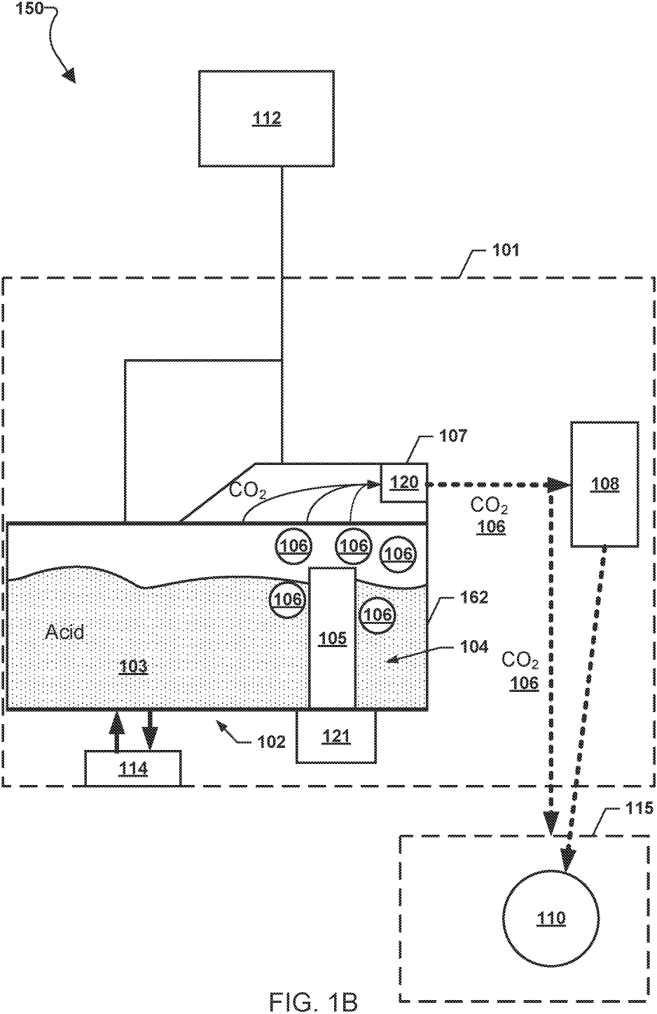

FIG. 1A illustrates an example system 100 according to various embodiments. The system 100 may include an apparatus 101 including a reactor 102 and a subreactor 104. The reactor 102 may be configured to produce at least an acid 103. The subreactor 104 may be configured for the dissolution of a metal carbonate 105 in the acid 103. In the embodiment illustrated in FIG. 1A the reactor 102 and subreactor 104 may be separate devices. The reactor 102 and subreactor 104 may be fluidically coupled, such as by a piping system or other fluid transfer system, and acid 103 produced by the reactor 102 may be provided to the subreactor 104. The apparatus 101 may include an apparatus 114 for storing acid 103, such as an acid storage tank, etc. The apparatus 114 for storing acid 103 may be fluidically coupled to the reactor 102 and/or the subreactor 104, such as by a piping system or other fluid transfer system.

In various embodiments, the reactor 102 may be any type reactor configured to produce an acid. In some embodiments, the reactor 102 may be a chemical reactor. When the reactor 102 is a chemical reactor, a chemical reaction may be used to produce acid 103 that is reacted with natural minerals or synthesized or waste materials containing metal carbonates 105 (e.g., limestone or dolomite) to release bound CO₂ 106. In some embodiments, the reactor 102 may be an electrochemical reactor. When the reactor 102 is an electrochemical reactor, electrochemistry may be used to produce acid 103 that is reacted with natural minerals or synthesized or waste materials containing metal carbonates 105 (e.g., limestone or dolomite) to release bound CO₂ 106. In certain embodiments, the reactor 102 may be an electrolytic reactor such as an electrolyzer, a chlor-alkali reactor, an electrodialysis unit, or other such electrochemical reactor that produces at least an acid 103. In some embodiments, the acid 103 is produced as an aqueous solution by the reactor 102 and collected for use in dissolving components of the metal carbonate 105.

In various embodiments, the metal carbonate 105 may be introduced to the subreactor 104 by a metal carbonate delivery mechanism 121, such as a pumping system, a mechanical delivery system, etc. The metal carbonate delivery mechanism 121 may be a subsystem of the subreactor 104.

In various embodiments, a collection system 107, such as a hood, vent system, cover, etc., may collect the CO₂ 106. For example, the collection system 107 may include a pumping and/or compression system 120 configured draw in the CO₂ 106 and move the CO₂ 106 to other fluidically connected devices, such as a CO₂ storage tank 108, a facility 115 containing one or more living organisms 110, or any other type storage system and/or operating system to which the CO₂ 106 may be applied to and/or used by. The collection system 107 may collect the CO₂ 106 produced upon dissolution of the metal carbonate 105 in the acid 103. In various embodiments, the pumping and/or compression system 120 may compress the CO₂ 106. In various embodiments, the subreactor 104 may include a pressure vessel 122 configured to operate such that the CO₂ 106 is produced within the pressure vessel 122 under a pressure greater than the pressure outside the pressure vessel 122, such as a pressure above one atmosphere, above two atmospheres, above three atmospheres, greater than three atmospheres, etc.

The system 100 may include a power source 112 providing power to the system 100. In some embodiments in which the reactor 102 is an electrochemical reactor, the power source 112 may provide electricity to the electrochemical reactor. In some embodiments, the power source 112 may be a renewable electricity source, such as a wind farm, solar plant, etc. In various embodiments, the power source 112 may provide power to other systems of the apparatus 101, such as the collection system 107, subreactor 104, etc.

In the system 100, the apparatus 101 may provide the CO₂ 106 produced upon dissolution of the metal carbonate 105 in the acid 103 to the facility 115 containing one or more living organisms 110. As examples, the one or more living organisms 110 may be animals, photosynthetic bacteria, agricultural products (e.g., food for humans, food for animals, etc.), plants (e.g., algae, another type of plant, etc.), and/or any other type living organisms. In various embodiments, the CO₂ 106 produced upon dissolution of the metal carbonate 105 in the acid 103 may be used to improve a health, a grow rate, and/or a yield of the one or more living organisms 110. In some embodiments, the one or more living organisms 110 may be plants used to produce a biofuel (e.g., algae used to produce a biofuel or other type plants used to produce a biofuel) and/or plants used for food (e.g., algae used to produce a food or other type plants used to produce a food). In various embodiments, the facility 115 may be used for farming and/or biofuel production. In various embodiments, the facility 115 may be an indoor farm, a greenhouse, and/or a biofuel production facility. In various embodiments, the facility 115 may be a medical care facility. In various embodiments, the facility 115 may be an agricultural system and/or aquaculture system.

FIG. 1B illustrates another example system 150 according to various embodiments. The system 150 may be similar to system 100, except that in system 150 the subreactor 104 may be a region within the reactor 102. For example, the subreactor 104 may be included in the reactor 102. In such embodiments in which the subreactor 104 is included in the reactor 102 (e.g., the subreactor 104 is a region of the reactor 102), the pressure vessel 162 may encompass the reactor 102. The pressure vessel 162 may be configured to operate such that the CO₂ 106 is produced within the pressure vessel 162 under a pressure greater than the pressure outside the pressure vessel 162, such as a pressure above one atmosphere, above two atmospheres, above three atmospheres, greater than three atmospheres, etc. The operation of system 150 may be similar to the operation of system 100, except that one region of the reactor 102 may generate the acid 103 while another region of the reactor 102, specifically the subreactor 104, produces the CO₂ 106 produced upon dissolution of the metal carbonate 105 in the acid 103.

In some embodiments, the CO₂ (e.g., CO₂ 106) the is used in industry. Non-limiting examples of CO₂ (e.g., CO₂ 106) use in industry include use as an inert gas for chemical processes, welding, as a lasing medium, preventing spoilage of foods and other air-sensitive materials, to extinguish fires, to dilute flammable or toxic vapours, as a non-reactive cooling gas.

Non-limiting examples of CO₂ (e.g., CO₂ 106) use in industry include use as a toxic gas to subdue animals before slaughter, to asphyxiate animals, to kill pests and rodents, or to bait and trap insects such as mosquitos and bedbugs.

Non-limiting examples of CO₂ (e.g., CO₂ 106) use in industry include use as a medicinal gas, for example CO₂ can be blended with oxygen for medicinal purposes, to stimulate breathing after apnea and stabilize O₂/CO₂ ratios in blood.

Non-limiting examples of CO₂ (e.g., CO₂ 106) use in industry include use as a food additive, propellant for food stuffs, acidity regulator in food, to add carbonation to sparkling beverages, and/or to prevent spoilage of food.

Non-limiting examples of CO₂ (e.g., CO₂ 106) use in industry include use as a reagent in industry, for example in the synthesis of urea, methanol, ethanol, synthetic fuels, carbonates (such as precipitated calcium carbonate), carboxylic acid derivatives, proteins, polymers, foams and aerogels.

Non-limiting examples of CO₂ (e.g., CO₂ 106) use in industry include to make building materials, for example using CO₂ to carbonate minerals, using CO₂ to carbonate waste materials (e.g., slag) to make carbonate rocks for use as aggregates in concrete, using CO₂ to promote curing of cementitious materials through carbonation of minerals in the cement paste, using CO₂ as an additive in cement pastes.

Non-limiting examples of CO₂ (e.g., CO₂ 106) use in industry include use for pest control and promoting plant growth in agriculture. For example, CO₂ may be used by indoor farms and greenhouses to enrich the air and promote photosynthesis and plant growth, and also to eliminate pests such as whiteflies and spidermites. As a further example, CO₂ can also be used to promote the growth of algaes, which later can be processed into bio fuels and other organic chemicals. As another example, CO₂ can also be used to control the pH of reef aquaria to promote the growth of corals, in the natural environment or in aquariums.

Non-limiting examples of CO₂ (e.g., CO₂ 106) use in industry include use for improving the productivity of oil wells. For example, carbon dioxide is injected into oil wells to enhance oil recovery. As an additional example, CO₂ can act as a pressurizing agent. As another example, CO₂ also becomes miscible in oil, reducing the oil’s viscosity and allowing it to flow more rapidly through pipes.

Non-limiting examples of CO₂ (e.g., CO₂ 106) use in industry include use as a propellant (e.g., for paintball guns), for pressure tools, for filling inflatable objects (e.g., life jackets, sports equipment, tires), for blasting (e.g., in coal mines), for loosening floor tiles.

Non-limiting examples of CO₂ (e.g., CO₂ 106) use in industry include use as a cleaning agent. The abrasive and propellant properties of solid CO₂ can be used for blast cleaning, an alternative to sandblasting, to clean materials and surfaces. CO₂ can be used to cool abrasives, reducing their elastic properties to facilitate removal. CO₂ can be used as a solvent to remove such things as ink, glue, oil, paint, and mould. Supercritical CO₂ can be used as a dry-cleaning solvent.

Non-limiting examples of CO₂ (e.g., CO₂ 106) use in industry include use as a solvent. CO₂, especially supercritical CO₂, can be used a solvent for lithophilic molecules. For example, it can be used to remove caffeine from coffee beans and as a dry cleaner for clothes.

Non-limiting examples of CO₂ (e.g., CO₂ 106) use in industry include use as a refrigerant. Solid CO₂ (dry ice) or supercritical CO₂ is used as a refrigerant, for the transportation of frozen or cooled foods, to flash-freeze food or biological supplies, solidify oil spills, to freeze and remove warts, to change the elastic properties of adhesives to facilitate removal. Solid CO₂ sublimes to create a fog in fog machines, used for aesthetic purposes (for example, at theatres, haunted houses, nightclubs), for freezing water in valveless pipes to enable repair, as a cutting fluid, as a means of condensing other gases and vapours.

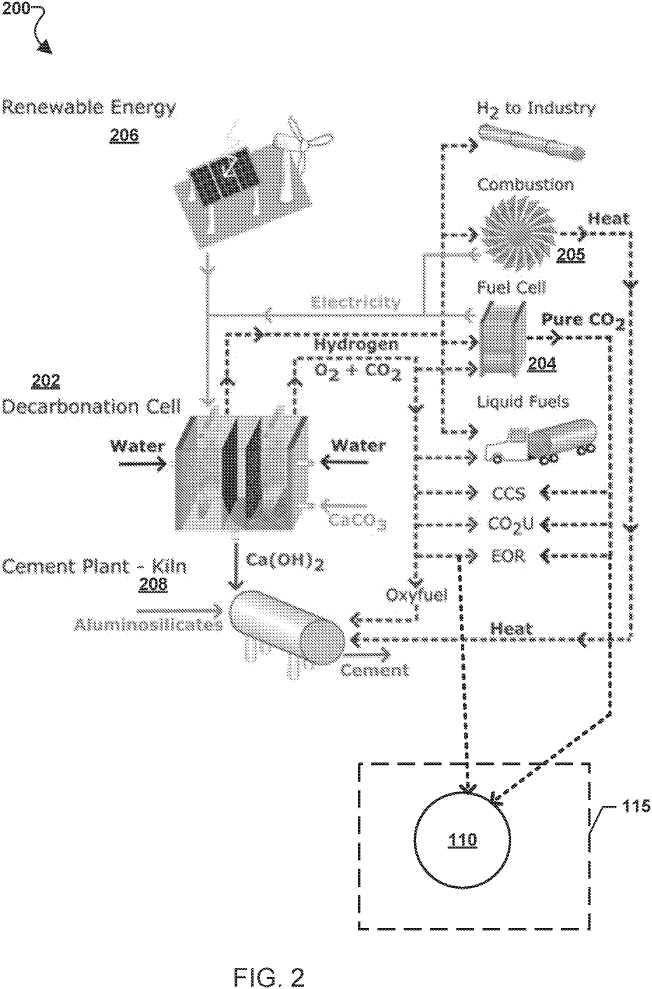

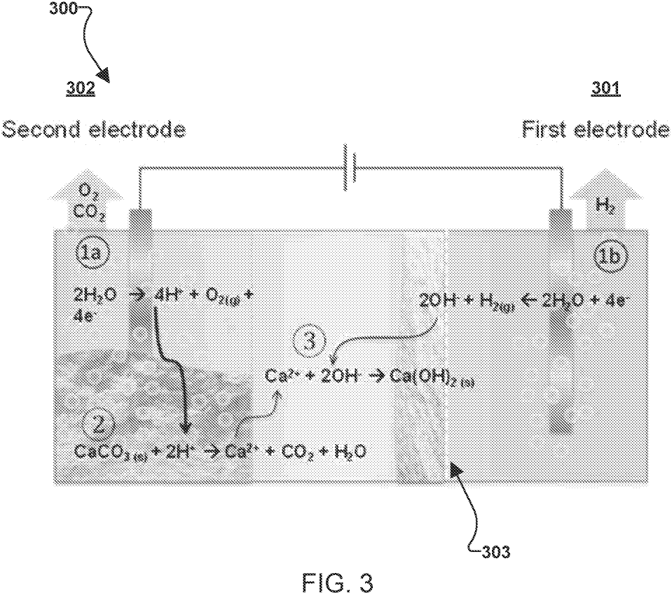

In one specific example of a system that generates CO₂ (such as a specific example of the system 150), a neutral-water electrolyzer is used to produce an acid stream at the oxygen-evolving cathode and an alkaline stream at the hydrogen-evolving anode, following the method described by L. D. Ellis, A. F. Badel, M. L. Chiang, R. J.-Y. Park, Y.-M. Chiang, “Towards Electrochemical Synthesis of Cement — An Electrolyzer-Based Process for Decarbonating CaCO₃ While Producing Useful Gas Streams,” PNAS, September 2019, 201821673; DOI: 10.1073/pnas.1821673116, the entire contents of which is fully incorporated herein for all purposes. Specifically, FIG. 2 illustrates a specific example system 200 that generates CO₂ (e.g., CO₂ 106) according to various embodiments. As an example, the system 200 may be a specific implementation of a system 150 described above with reference to FIG. 1B. For example, the reactor 102 may be a neutral-water electrolyzer 202 and the power source 112 may be a renewable energy power source 206 (e.g., providing electricity from wind energy, solar energy, etc.). As a specific example, the neutral-water electrolyzer 202 may be an electrochemical reactor 300 as illustrated in FIG. 3. As illustrated in FIG. 2, the electrochemical decarbonation reactor (decarbonation cell 202) powered by renewable electricity from renewable energy source 206 converts CaCO₃ to Ca(OH)₂ for use in cement synthesis by a cement plant kiln 208. The decarbonation cell 202 uses the pH gradient produced by neutral-water electrolysis to dissolve CaCO₃ at the acidic anode and precipitate Ca(OH)₂ where the pH ≥ 12.5. Simultaneously, H₂ is generated at the cathode and O₂/CO₂ are generated at the anode. These gas streams can serve several alternative roles in a sustainable production system. CO₂ can be directly captured for carbon capture and sequestration (CCS). Electricity or heat can be generated from the H₂ and O₂ via fuel cells 204 or combustors 205. The O₂/CO₂ oxy-fuel can be recirculated to the kiln 208 for cleaner combustion in the cement sintering cycle. CO₂ reuse and utilization (CO₂U) concepts can be employed, such as use in enhanced oil recovery (EOR) or production of liquid fuels. Additionally, CO₂ may be provided to the facility 115 and one or more living organisms 110 as discussed herein (either as part of the O₂/CO₂ stream or as pure CO₂ from the fuel cell 204). The acidic solution from the electrolyser 202 is used to selectively dissolve at least certain carbonate salts present, resulting in the evolution of CO₂. The basic solution from the electrolyser 202 is used to precipitate hydrated lime (Ca(OH)₂). In this embodiment, the CO₂ may be mixed with O₂ and H₂O gases.

In some embodiments, the invention provides an increased purity of the CO₂ (e.g., CO₂ 106) compared to that which is obtained by other means (e.g., capture from flue gases). In some embodiments, an advantage of the invention is the reduced carbon footprint of the CO₂ (e.g., CO₂ 106) that is produced, since it is not derived from fossil fuel or combustion. In some embodiments, an advantage of the invention is the co-production of hydrogen gas, oxygen gas, or precipitated metal hydroxides. A non-limiting example of an advantage of co-production is in agriculture, where CO₂ (e.g., CO₂ 106) is used to promote the growth of plants, and calcium hydroxide (lime) is used to reduce acidity of soil and also promote the growth of plants. A non-limiting example of an advantage of co-production is in the fabrication of synthetic fuels, where CO₂ (e.g., CO₂ 106) is reacted with H₂. A non-limiting example of an advantage of co-production is in the synthesis of precipitated calcium carbonate or the carbonation of cements that are made from Ca(OH)₂.

In some embodiments, the acidic solution comprises acid (e.g., acid produced in the reactor (e.g., acid 103)). According to certain embodiments, the pH of the acidic solution is less than 7, less than or equal to 6, less than or equal to 5, less than or equal to 4, less than or equal to 3, less than or equal to 2, less than or equal to 1, or less than or equal to 0.

In some embodiments, the temperature of one or more of the dissolution step(s) and/or precipitation step(s) may each independently be greater than or equal to -10° C., greater than or equal to -5° C., greater than or equal to 0° C., greater than or equal to 5° C., greater than or equal to 10° C., greater than or equal to 15° C., greater than or equal to 20° C., greater than or equal to 30° C., greater than or equal to 40° C., or greater than or equal to 50° C. In certain embodiments, the temperature of one or more of the dissolution step(s) and/or precipitation step(s) may each independently be less than or equal to 100° C., less than or equal to 90° C., less than or equal to 80° C., less than or equal to 70° C., less than or equal to 60° C., less than or equal to 50° C., less than or equal to 40° C., less than or equal to 30° C., less than or equal to 25° C., less than or equal to 20° C., less than or equal to 15° C., less than or equal to 10° C., less than or equal to 5° C., or less than or equal to 0° C. In some embodiments, the temperature of one or more of the dissolution step(s) and/or precipitation step(s) may be room temperature. Combinations of these ranges are also possible (e.g., greater than or equal to -10° C. and less than or equal to 50° C., greater than or equal to -5° C. and less than or equal to 10° C., greater than or equal to 15° C. and less than or equal to 25° C., or greater than or equal to 50° C. and less than or equal to 100° C.).

In some cases, agitation (e.g., stirring, sonication, and/or shaking) affects the solubility of the various substances and/or components. In certain instances, one or more of the dissolution step(s) and/or precipitation step(s) comprises agitation.

In certain embodiments, the dissolution of the carbonate yielding CO₂ gas occurs inside the reactor. In some embodiments, the method comprises collecting and/or storing the acid and/or the base produced in the reactor. According to certain embodiments, the dissolution step(s) occur outside the reactor.

In some embodiments, the system (e.g., system 100, 150, 200, etc.), and as a specific example the reactor (e.g., reactor 102, 202, 300), is powered at least in part (e.g., at least 10%, at least 25%, at least 50%, at least 75%, at least 90%, or 100%) by renewable electricity (e.g., solar energy and/or wind energy).

In some embodiments, the system (e.g., system 100, 150, 200, etc.), and as a specific example the reactor (e.g., reactor 102, 202, 300), comprises a first electrode. In some embodiments, the first electrode comprises a cathode. In certain embodiments, the first electrode is selected to be an electronic conductor that is stable under relatively alkaline conditions (e.g., in an alkaline region and/or base described herein). In certain embodiments, the first electrode comprises a metallic electrode (such as platinum, gold, nickel, iridium, copper, iron, steel, stainless steel, manganese, and/or zinc), carbon (such as graphite or disordered carbons), or a metal carbide (such as silicon carbide, titanium carbide, and/or tungsten carbide). In certain embodiments, the first electrode comprises a metal alloy (e.g., a nickel-chromium-iron alloy, nickel-molybdenum-cadmium alloy), a metal oxide (e.g., iridium oxide, nickel iron cobalt oxide, nickel cobalt oxide, lithium cobalt oxide, lanthanum strontium cobalt oxide, barium strontium ferrous oxide, manganese molybdenum oxide, ruthenium dioxide, iridium ruthenium tantalum oxide), a metal organic framework, or a metal sulfide (e.g., molybdenum sulfide). In certain embodiments, electrocatalyst or electrode material is dispersed or coated onto a conductive support.

In some embodiments, the system (e.g., system 100, 150, 200, etc.), and as a specific example the reactor (e.g., reactor 102, 202, 300), comprises a second electrode. In some embodiments, the second electrode comprises an anode. In some embodiments, the second electrode is electrochemically coupled to the first electrode. That is to say, the electrodes can be configured such that they are capable of participating in an electrochemical process. Electrochemical coupling can be achieved, for example, by exposing the first and second electrodes to an electrolyte that facilitates ionic transport between the two electrodes. In certain embodiments, the second electrode is selected to be an electronic conductor that is stable under relatively acidic conditions (e.g., in an acidic region and/or acid described herein). In certain embodiments, the second electrode comprises a metallic electrode (such as platinum, palladium, lead, and/or tin) or a metal oxide (such as a transition metal oxide).

In certain embodiments, the first electrode and/or the second electrode comprise catalysts. In some embodiments the cathode catalyst is selected to be stable under alkaline conditions. The cathode catalyst can comprise, in some embodiments, nickel, iron, a transition metal sulfide (such as molybdenum sulfide), and/or a transition metal oxide (such as MnO₂, Mn₂O₃, Mn₃O₄, nickel oxide, nickel hydroxide, iron oxide, iron hydroxide, cobalt oxide), a mixed transition metal spinel oxide (such as MnCo₂O₄, CoMn₂O₄, MnFe₂O₄, ZnCoMnO₄), and the like. In some embodiments the anode catalyst is selected to be stable under acidic conditions. In some embodiments, the anode catalyst comprises platinum, iridium or their oxides.

In some embodiments, the system comprises a reactor (e.g., an electrochemical reactor or other type reactor). In some embodiments, the reactor comprises the first electrode and the second electrode. For example, in some embodiments, the first electrode is electrochemically coupled to the second electrode in the reactor. FIG. 3 illustrates an example of such a reactor 300 including a first electrode 301 and the second electrode 302.

In some embodiments, the method comprises running a reactor (e.g., any reactor described herein). In certain cases, running the reactor comprises applying current to an electrode of the reactor. In some embodiments, running the reactor results in at least one chemical reaction occurring within the reactor.

In certain embodiments, the method comprises running a reactor in a first mode. In some embodiments, the first mode comprises producing base near the first electrode (e.g., base is produced as a result of an electrochemical reaction in the first electrode). In certain embodiments, the first electrode (e.g., in the first mode) is configured to produce a basic output (e.g., any of the bases described herein).

The base may have any of a variety of suitable concentrations. In some embodiments, the base has a concentration of greater than or equal to 0.000001 M, greater than or equal to 0.00001 M, greater than or equal to 0.0001 M, greater than or equal to 0.001 M, greater than or equal to 0.01 M, greater than or equal to 0.1 M, greater than or equal to 0.5 M, greater than or equal to 1 M, greater than or equal to 3 M, greater than or equal to 5 M, greater than or equal to 7 M, greater than or equal to 10 M, greater than or equal to 15 M, or greater than or equal to 20 M. In certain embodiments, the base has a concentration of less than or equal to 25 M, less than or equal to 20 M, less than or equal to 15 M, less than or equal to 10 M, less than or equal to 7 M, less than or equal to 5 M, or less than or equal to 3 M. Combinations of these ranges are also possible (e.g., greater than or equal to 0.1 M and less than or equal to 25 M or greater than or equal to 0.1 M and less than or equal to 10 M).

In accordance with some embodiments, the production of the base by the first electrode (e.g., 301) results in an alkaline region (e.g., any alkaline region described herein) near the first electrode (e.g., within the half of the reactor compartment that is closest to the first electrode). For example, in some instances, the fluid adjacent the first electrode (e.g., the alkaline region) has a higher pH than fluid further away from the first electrode.

In some embodiments, the pH near (e.g., adjacent to) the first electrode (e.g., 301) is greater than 7, greater than or equal to 8, greater than or equal to 9, greater than or equal to 10, greater than or equal to 11, greater than or equal to 12, greater than or equal to 13, or greater than or equal to 14. In accordance with some embodiments, the pH near the first electrode (e.g., 301) is less than or equal to 19, less than or equal to 16, less than or equal to 14, less than or equal to 13, less than or equal to 12, less than or equal to 11, or less than or equal to 10. Combinations of these ranges are also possible (e.g., greater than 7 and less than or equal to 19, greater than or equal to 9 and less than or equal to 16, greater than or equal to 8 and less than or equal to 14).

In some embodiments, the second electrode (e.g., 302) is configured to produce an acidic output (e.g., any of the acids described herein). In certain embodiments, the acidic output is produced as a result of an electrochemical reaction in the second electrode. In some embodiments, the first mode of the reactor comprises producing acid near the second electrode (e.g., acid is produced as a result of an electrochemical reaction in the second electrode).

The acid may have any of a variety of suitable concentrations. In some embodiments, the acid has a concentration of greater than or equal to 0.000001 M, greater than or equal to 0.00001 M, greater than or equal to 0.0001 M, greater than or equal to 0.001 M, greater than or equal to 0.01 M, greater than or equal to 0.1 M, greater than or equal to 0.5 M, greater than or equal to 1 M, greater than or equal to 3 M, greater than or equal to 5 M, greater than or equal to 7 M, or greater than or equal to 10 M. In certain embodiments, the acid has a concentration of less than or equal to 12 M, less than or equal to 10 M, less than or equal to 7 M, less than or equal to 5 M, less than or equal to 3 M, or less than or equal to 1 M. Combinations of these ranges are also possible (e.g., greater than or equal to 0.000001 M and less than or equal to 12 M or greater than or equal to 0.1 M and less than or equal to 10 M).

In accordance with some embodiments, the production of the acid by the second electrode (e.g., 302) results in an acidic region (e.g., any acidic region described herein) near the second electrode (e.g., within the half of the reactor compartment that is closest to the second electrode). For example, in some instances, the fluid adjacent the second electrode (e.g., the acidic region) has a lower pH than fluid further away from the second electrode. As an example, in some cases, the system comprises acidic region near second electrode (e.g., 302).

According to certain embodiments, the pH near (e.g., adjacent to) the second electrode (e.g., 302) has a pH of less than 7, less than or equal to 6, less than or equal to 5, less than or equal to 4, less than or equal to 3, less than or equal to 2, less than or equal to 1, or less than or equal to 0. In some embodiments, the pH near the second electrode has a pH of greater than or equal to -5, greater than or equal to -2, greater than or equal to 0, greater than or equal to 1, greater than or equal to 2, greater than or equal to 3, greater than or equal to 4, or greater than or equal to 5. Combinations of these ranges are also possible (e.g., greater than or equal to -5 and less than 7, greater than or equal to -2 and less than or equal to 1, greater than or equal to 0 and less than or equal to 6).

In certain embodiments, the first electrode (e.g., cathode (e.g., 301)) is configured to produce hydrogen gas, such that hydrogen gas can be produced near the first electrode (e.g., the hydrogen gas is produced as a result of an electrochemical reaction in the first electrode). In some instances, running the reactor in the first mode comprises producing hydrogen gas (e.g., hydrogen gas and base) near the first electrode (e.g., hydrogen gas is produced as a result of an electrochemical reaction in the first electrode). In some instances, the hydrogen gas and/or base are produced near the first electrode by reduction of water near the first electrode.

In certain embodiments, the second electrode (e.g., anode (e.g., 302)) is configured to produce oxygen, such that oxygen gas can be produced near the second electrode (e.g., the oxygen gas is produced as a result of an electrochemical reaction in the second electrode). In certain cases, running the reactor in the first mode comprises producing oxygen gas (e.g., oxygen gas and acid) near the second electrode (e.g., oxygen gas is produced as a result of an electrochemical reaction in the second electrode). In some instances, the oxygen gas and/or acid are produced near the second electrode by oxidation of water near the second electrode.

In some embodiments, the system is configured to allow oxygen gas to diffuse and/or be transported to a location near the first electrode (e.g., 301) (e.g., from a location near the second electrode (e.g., 302)). For example, in some cases, the system is configured to allow oxygen gas to diffuse and/or be transported to fluid near the first electrode (e.g., 301), such that the oxygen gas could be involved in an electrochemical reaction in the first electrode, from fluid near the second electrode, after the oxygen gas was produced as a result of an electrochemical reaction in the second electrode.

According to certain embodiments, the system is configured to allow the oxygen gas to be reduced near the first electrode (e.g., 301) (e.g., the oxygen gas is reduced as a result of an electrochemical reaction in the first electrode). In some embodiments, reducing the oxygen gas near the first electrode comprises production of a base. In certain embodiments, the production of a base is advantageous because it increases the overall amount of base produced at the first electrode.

In some embodiments, the system is configured to allow hydrogen gas to diffuse and/or be transported to a location near the second electrode (e.g., 302) (e.g., from a location near the first electrode (e.g., 301)). For example, in some cases, the system is configured to allow hydrogen gas to diffuse and/or be transported to fluid near the second electrode, such that the hydrogen gas could be involved in an electrochemical reaction in the second electrode, from fluid near the first electrode, after the hydrogen gas was produced as a result of an electrochemical reaction in the first electrode.

According to certain embodiments, the system is configured to allow the hydrogen gas to be oxidized near the second electrode (e.g., 302) (e.g., hydrogen gas is oxidized as a result of an electrochemical reaction in the second electrode). In some embodiments, oxidizing the hydrogen gas near the second electrode comprises production of acid. In certain embodiments, the production of acid is advantageous because it increases the overall amount of acid produced at the second electrode.

In some embodiments, the system comprises a separator (e.g., 303). In certain embodiments, the separator is configured to allow oxygen gas produced at the second electrode (e.g., 302) to diffuse to the first electrode (e.g., 301) and/or to allow hydrogen gas produced at the first electrode to diffuse to the second electrode. For example, in some embodiments, the separator is permeable to oxygen gas and/or hydrogen gas.

There may be many suitable ways to transport the hydrogen gas and/or oxygen gas from one electrode to the other. In certain embodiments, the hydrogen gas and/or oxygen gas could be transported via a conduit (e.g., a pipe, channel, needle, or tube). In some cases, the hydrogen gas and/or oxygen gas could be transported directly from one electrode to another, or the hydrogen gas and/or oxygen gas could be stored after removal from the reactor until it is added back into the reactor. In some embodiments, the hydrogen gas and/or oxygen gas is transported continuously or in batches. In certain embodiments, the hydrogen gas and/or oxygen gas is transported automatically or manually.

In some embodiments, hydrogen gas produced by hydrolysis may be electrochemically oxidized using the hydrogen oxidation reaction (HOR) in which one dihydrogen molecule reacts to form two protons and two electrons. In other embodiments, oxygen gas produced by hydrolysis may be electrochemically reduced in the oxygen reduction reaction (ORR) wherein one dioxygen molecule reacts with two water molecules and four electrons to form four hydroxyl ions. In some embodiments, the HOR reaction is used to lower the pH or increase the proton concentration of the acidic solution produced by the reactor. In some embodiments, the ORR reaction is used to increase the pH or increase the hydroxyl concentration of the basic solution produced by the reactor. HOR and ORR reactions as herein described may be carried out, in some cases, using separate electrodes from those used for the electrolysis reaction of the reactor. In certain embodiments, these electrodes may be located within the electrolysis reactor, for example, as a combustion electrode where the hydrogen and oxygen combustion reaction produces water that remains within the reactor. The electrodes used for combustion, or for HOR or ORR, may, in some instances, also be located in a separate vessel or reactor, to which the hydrogen or oxygen gas is each delivered. In some embodiments, the hydrogen produced at the cathode of the electrolysis reactor is delivered to an HOR electrode connected to the anode side of the reactor, where HOR is conducted and the protons produced thereby increase the acid concentration (lowering the pH) of the acidic solution that is produced by the reactor. In certain embodiments, the oxygen produced at the anode of the electrolysis reactor is delivered to an ORR electrode connected to the cathode side of the reactor, where ORR is conducted and the hydroxyl ions produced thereby increase the hydroxyl concentration (increasing the pH) of the alkaline solution that is produced by the reactor. In some instances, the HOR reaction is preferentially conducted over the ORR reaction to reduce the release of hydrogen as compared to the less reactive oxygen to the external environment. The electrodes used for hydrogen-oxygen combustion or HOR or ORR may, in some cases, comprise compounds that function as electrocatalysts. Hydrogen-oxygen combustion catalysts have been studied. Examples of electrocatalysts for HOR and ORR include platinum group metals such as Pt, Pd, Ru, Rh, Os, and Ir, non-platinum group metals such as Mo, Fe, Ti, W, Cr, Co, Cu, Ag, Au, and Re, used individually or as alloys or mixtures; high surface area nickel-aluminum alloys known as Raney nickel, optionally coated or doped with other catalysts. Examples of electrocatalysts selective for ORR include metallic iron, iron oxides, iron sulfide, and iron hydroxide, silver alloys, oxides and nitrate, and various forms of carbons including carbon paper, carbon felt, graphite, carbon black, and nanoscale carbons.

In certain embodiments described herein, the non-CO₂ gaseous byproducts produced by electrolysis (e.g., H₂ and/or O₂) may have value and may be sold for use in other applications and processes, including combustion in a fuel cell or gas turbine or internal combustion engine for the purpose of producing energy and power, including electric power. However, in some instances, it may be desirable to reduce or eliminate the production of such gases. Accordingly, in some embodiments, one or more of the gases produced by the reactor are recombined. As used herein, recombination refers to chemical or electrochemical reactions that consume one or more of the gases produced.

In some embodiments, hydrogen and oxygen produced by hydrolysis are recombined using hydrogen-oxygen combustion to form water. In accordance with certain embodiments, hydrogen-oxygen recombination may take place within or external to the reactor, and may, in some cases, use electrode materials and designs, and optionally catalysts, well-known to those skilled in the art. In certain embodiments, the method does not produce net hydrogen gas (or the net amount of hydrogen gas produced is less than 5% (e.g., less than 2% or less than 1%) of the current supplied to the reactor). For example, in some embodiments, the method does not release any hydrogen gas (or the amount of hydrogen gas released is less than 5% (e.g., less than 2% or less than 1%) of the current supplied to the reactor) to the atmosphere, as the hydrogen gas produced is recombined with oxygen to form water. Similarly, in some cases, the method does not produce net oxygen gas (or the net amount of oxygen gas produced is less than 5% (e.g., less than 2% or less than 1%) of the current supplied to the reactor). For example, in certain instances, the method does not release any oxygen gas (or the net amount of oxygen gas released is less than 5% (e.g., less than 2% or less than 1%) of the current supplied to the reactor) to the atmosphere, as the oxygen gas produced is recombined with hydrogen to form water.

In some embodiments, hydrolysis is carried out under conditions that produce a basic pH near the first electrode (e.g., the cathode (e.g., 301)), and an acidic pH near the second electrode (e.g., the anode (e.g., 302)), without liberating hydrogen gas or oxygen gas (or the amount of hydrogen gas or oxygen gas liberated is less than 5% (e.g., less than 2% or less than 1%) of the current supplied to the reactor), respectively. For example, in some embodiments, O₂ could diffuse (e.g., through the electrolyte and/or through air above the electrolyte) from the second electrode (e.g., anode (e.g., 302)), where acid and O₂ are produced, to the first electrode (e.g., the cathode (e.g., 301)), where base is produced and where the O₂ would be reduced to form OH⁻ (½ O₂ + H₂O + 2e⁻ ➔ 2 OH⁻). In certain embodiments, this reaction would occur at pH > 7 and an electrode potential less than 0.8 V vs the standard hydrogen electrode. Similarly, in some cases, H₂ could diffuse from the first electrode (e.g., the cathode (e.g., 301)), where base is produced, to the second electrode (e.g., the anode (e.g., 302)), where acid is produced and where the H₂ would be oxidized to form H⁺ (H₂ ➔ 2H⁺ + 2e⁻). In certain instances, this would occur when the pH is <7 and when the electrode potential is greater than -0.41 V vs the standard hydrogen electrode. In other electrolyzers, such as an alkaline electrolyzer, this reaction is hindered by a separator that prevents the crossover of gases between the two electrodes. However, in some embodiments disclosed herein, the reactor comprises a separator that allows and/or promotes crossover of H₂ and/or O₂, such that they can be consumed and increase the pH gradient.

In some embodiments, acidic solutions (less than pH 7) are generated from neutral-pH electrolytes at electrode potentials greater than 0.8 V vs the standard hydrogen electrode. For example, in certain embodiments, to make an acidic solution of pH 0, the minimum electrode potential would be 1.23 V vs the standard hydrogen electrode. In some cases, basic solutions (greater than pH 7) are generated from neutral-pH electrolytes at electrode potentials less than -0.4 V vs the standard hydrogen electrode. For example, to make an alkaline solution of pH 14, the maximum electrode potential would be -0.83 V vs the standard hydrogen electrode.

The Nernst potential at the second electrode (e.g., 302) (e.g., the Nernst potential in the fluid nearest the second electrode) may be any of a variety of suitable values. In some embodiments, the Nernst potential at the second electrode (e.g., the anode) is greater than or equal to -0.4 V, greater than or equal to -0.2 V, greater than or equal to 0 V, greater than or equal to 0.5 V, greater than or equal to 0.8 V, greater than or equal to 0.9 V, greater than or equal to 1 V, greater than or equal to 1.1 V, greater than or equal to 1.2 V, greater than or equal to 1.4 V, or greater than or equal to 1.6 V vs the standard hydrogen electrode. In certain embodiments, the Nernst potential at the second electrode is less than or equal to 2 V, less than or equal to 1.7 V, less than or equal to 1.5 V, less than or equal to 1.4 V, less than or equal to 1.3 V, less than or equal to 1.2 V, less than or equal to 1.1 V, less than or equal to 1 V, less than or equal to 0.9 V, less than or equal to 0.8 V, less than or equal to 0.5 V, less than or equal to 0 V, or less than or equal to -0.2 V vs the standard hydrogen electrode. Combinations of these ranges are also possible (e.g., greater than or equal to 0.8 V and less than or equal to 2 V, greater than or equal to 1.2 V and less than or equal to 2 V, greater than or equal to -0.4 V and less than or equal to 0.5 V, or greater than or equal to 0 V and less than or equal to 0.5 V).

In certain embodiments, the suitable Nernst potential at the second electrode (e.g., 302) depends on the type of reaction at the electrode. For example, in some cases, the Nernst potential at the second electrode when hydrogen gas is oxidized to acid is greater than or equal to -0.4 V vs the standard hydrogen electrode (e.g., greater than or equal to -0.4 V and less than or equal to 0.5 V or greater than or equal to 0 V and less than or equal to 0.5 V). As another example, in certain instances, the Nernst potential at the second electrode when water is oxidized to acid and oxygen gas is greater than or equal to 0.8 V vs the standard hydrogen electrode (e.g., greater than or equal to 0.8 V and less than or equal to 2 V or greater than or equal to 1.2 V and less than or equal to 2 V).

The Nernst potential at the first electrode (e.g., 301) (e.g., the Nernst potential in the fluid nearest the first electrode) may be any of a variety of suitable values. In certain embodiments, the Nernst potential at the first electrode (e.g., cathode (e.g., 301)) is less than or equal to 0.8 V, less than or equal to 0.6 V, less than or equal to 0.4 V, less than or equal to 0 V, less than or equal to -0.4 V, less than or equal to -0.5 V, less than or equal to -0.6 V, less than or equal to -0.7 V, less than or equal to -0.8 V, less than or equal to -0.9 V, less than or equal to -1 V, less than or equal to -1.2 V, or less than or equal to -1.4 V vs the standard hydrogen electrode. In some embodiments, the Nernst potential at the first electrode is greater than or equal to -2 V, greater than or equal to -1.7 V, greater than or equal to -1.5 V, greater than or equal to -1.2 V, greater than or equal to -1 V, greater than or equal to -0.9 V, greater than or equal to -0.8 V, greater than or equal to -0.7 V, greater than or equal to -0.6 V, greater than or equal to -0.5 V, greater than or equal to -0.4 V, greater than or equal to 0 V, greater than or equal to 0.4 V, or greater than or equal to 0.6 V vs the standard hydrogen electrode. Combinations of these ranges are also possible (e.g., greater than or equal to -1.5 V and less than or equal to -0.4 V, greater than or equal to -1.5 V and less than or equal to -0.8 V, greater than or equal to -0.4 V and less than or equal to 0.8 V, or greater than or equal to -0.4 V and less than or equal to 0.4 V).

In certain embodiments, the suitable Nernst potential at the first electrode (e.g., 301) depends on the type of reaction at the electrode. For example, in some cases, the Nernst potential at the first electrode when oxygen gas is reduced to base is less than or equal to 0.8 V vs the standard hydrogen electrode (e.g., less than or equal to 0.8 V and greater than or equal to -0.4 V or less than or equal to 0.4 V and greater than or equal to -0.4 V). As another example, in certain instances, the Nernst potential at the first electrode when water is reduced to base and hydrogen gas is less than or equal to -0.4 V vs the standard hydrogen electrode (e.g., less than or equal to -0.4 V and greater than or equal to -1.5 V, or less than or equal to -0.8 V and greater than or equal to -1.5 V).

In certain embodiments, the cell voltage (e.g., the voltage applied to the cell, for example, during production of acid and/or base) is greater than or equal to 0 V, greater than or equal to 0.5 V, greater than or equal to 1 V, greater than or equal to 1.23 V, greater than or equal to 1.5 V, greater than or equal to 2 V, greater than or equal to 2.06 V, or greater than or equal to 2.5 V vs the standard hydrogen electrode. In some embodiments, the cell voltage is less than or equal to 5 V, less than or equal to 4 V, less than or equal to 3 V, less than or equal to 2.5 V, less than or equal to 2.25 V, less than or equal to 2 V, less than or equal to 1.5 V, less than or equal to 1 V, or less than or equal to 0.5 V vs the standard hydrogen electrode. Combinations of these ranges are also possible (e.g., 0-5 V or 0-2.5 V).

In some embodiments, the system (e.g., system 100, 150, 200, etc.) comprises a reactor system for producing concentrated acid and base. In accordance with some embodiments, the system comprises a first reactor (e.g., any reactor described herein). According to some embodiments, the system comprises a second reactor (e.g., any reactor described herein). In certain cases, the first reactor and the second reactor are fluidically connected. For instance, in some cases, a fluid (e.g., a liquid or a gas) produced in the first reactor can diffuse and/or be transported to the second reactor. As a non-limiting example, in certain embodiments, the method comprises diffusing and/or transporting hydrogen gas and/or dihalide from the first reactor to the second reactor.

In some embodiments, the first reactor comprises an electrochemical reactor. In certain cases, the first reactor comprises a first electrode (e.g., any first electrode described herein). In some embodiments, the second electrode is electrochemically coupled to the first electrode (e.g., the electrodes are configured such that current may flow from one electrode to the other). That is to say, the electrodes can be configured such that they are capable of participating in an electrochemical process. Electrochemical coupling can be achieved, for example, by exposing the first and second electrodes to an electrolyte that facilitates ionic transport between the two electrodes.

In certain instances, the second reactor comprises a fuel cell (e.g., an H₂/Cl₂ fuel cell). In some embodiments, the method comprises producing an acid in the second reactor.

In certain embodiments, the method comprises producing a base (e.g., any base described herein), a dihalide, and/or hydrogen gas in the first reactor. For example, in some cases, first reactor is configured to produce a base, a dihalide, and/or hydrogen gas. In some instances, the dihalide is produced near the second electrode of the first reactor (e.g., dihalide is produced as a result of an electrochemical reaction in the second electrode of the first reactor). For instance, in certain cases, dihalide is produced near second electrode of first reactor. In some embodiments, the base and/or hydrogen gas is produced near the first electrode (e.g., base and/or hydrogen gas is produced as a result of an electrochemical reaction in the first electrode). For example, in certain cases, base is produced near the first electrode (e.g., 301).

The Nernst potential at the second electrode of the first reactor (e.g., the Nernst potential in the fluid nearest the second electrode) may be any of a variety of suitable values. In some embodiments, the Nernst potential at the second electrode (e.g., the anode) of the first reactor is greater than or equal to 1.3 V, greater than or equal to 1.5 V, greater than or equal to 1.7 V, greater than or equal to 1.9 V, greater than or equal to 2.1 V, or greater than or equal to 2.3 V vs the standard hydrogen electrode. In certain embodiments, the Nernst potential at the second electrode of the first reactor is less than or equal to 2.5 V, less than or equal to 2.3 V, less than or equal to 2.1 V, less than or equal to 1.9 V, less than or equal to 1.7 V, or less than or equal to 1.5 V vs the standard hydrogen electrode. Combinations of these ranges are also possible (e.g., greater than or equal to 1.3 V and less than or equal to 2.5 V).

In certain embodiments, the suitable Nernst potential at the second electrode of the first reactor depends on the type of reaction at the electrode. For example, in some cases, the Nernst potential at the second electrode when dihalide is produced (e.g., chloride ions are being oxidized to form Cl₂) is greater than or equal to 1.3 V vs the standard hydrogen electrode (e.g., greater than or equal to 1.3 V and less than or equal to 2.5 V).

In some embodiments, Cl₂ is generated from Cl- at Nernst potentials above 1.36 V vs the standard hydrogen electrode (e.g., greater than or equal to 1.4 V, greater than or equal to 1.5 V, greater than or equal to 1.7 V, or greater than or equal to 2 V; less than or equal to 5 V, less than or equal to 3 V, less than or equal to 2 V, or less than or equal to 1.5 V; combinations are also possible) vs the standard hydrogen electrode.

In certain embodiments, Br₂ is generated from Br- at Nernst potentials greater than 1.06 V vs the standard hydrogen electrode (e.g., greater than or equal to 1.1 V, greater than or equal to 1.2 V, greater than or equal to 1.3 V, greater than or equal to 1.5 V, or greater than or equal to 1.8 V; less than or equal to 4 V, less than or equal to 3 V, less than or equal to 2 V, or less than or equal to 1.5 V; combinations are also possible) .

In some cases, I₂ is generated from I- at Nernst potentials greater than 0.54 V vs the standard hydrogen electrode (e.g., greater than or equal to 0.6 V, greater than or equal to 0.7 V, greater than or equal to 0.8 V, greater than or equal to 0.9 V, greater than or equal to 1 V, or greater than or equal to 1.2 V; less than or equal to 3 V, less than or equal to 2 V, less than or equal to 1.5 V, less than or equal to 1.3 V, or less than or equal to 1 V; combinations are also possible).

The Nernst potential at the first electrode of the first reactor (e.g., the Nernst potential in the fluid nearest the first electrode (e.g., 301)) may be any of a variety of suitable values. In some embodiments, the Nernst potential at the first electrode (e.g., the cathode (e.g., 301)) of the first reactor is greater than or equal to -2 V, greater than or equal to -1.8 V, greater than or equal to -1.6 V, greater than or equal to -1.4 V, greater than or equal to -1.2 V, greater than or equal to -1.0 V, greater than or equal to -0.8 V, greater than or equal to -0.6 V, greater than or equal to -0.4 V, greater than or equal to -0.2 V, greater than or equal to 0 V, greater than or equal to 0.2 V, greater than or equal to 0.4 V, or greater than or equal to 0.6 V vs the standard hydrogen electrode. In certain embodiments, the Nernst potential at the first electrode of the first reactor is less than or equal to 0.8 V, less than or equal to 0.6 V, less than or equal to 0.4 V, less than or equal to 0.2 V, less than or equal to 0 V, less than or equal to -0.2 V, less than or equal to -0.4 V, less than or equal to -0.6 V, less than or equal to -0.8 V, less than or equal to -1.0 V, less than or equal to -1.2 V, less than or equal to -1.4 V, or less than or equal to -1.6 V vs the standard hydrogen electrode. Combinations of these ranges are also possible (e.g., greater than or equal to -2 V and less than or equal to 0.8 V, greater than or equal to -1.4 V and less than or equal to 0.4 V, greater than or equal to -2 V and less than or equal to -0.4 V, or greater than or equal to -2 V and less than or equal to -0.8 V).

In certain embodiments, the suitable Nernst potential at the first electrode (e.g., 301) of the first reactor depends on the type of reaction at the electrode. For example, in some cases, the Nernst potential at the first electrode when oxygen is reduced to form base is less than or equal to 0.8 V vs the standard hydrogen electrode (e.g., greater than or equal to -2 V and less than or equal to 0.8 V or greater than or equal to -1.4 V and less than or equal to 0.4 V). As another example, in certain instances, the Nernst potential at the first electrode when water is reduced to hydrogen gas and base is less than or equal to -0.4 V vs the standard hydrogen electrode (e.g., greater than or equal to -2 V and less than or equal to -0.4 V, or greater than or equal to -2 V and less than or equal to -0.8 V).

In certain embodiments, the first reactor produces a base/alkaline solution, a dihalide, and hydrogen gas from an electrolyte containing a halide salt. In certain embodiments, a neutral water electrolyzer based reactor as disclosed herein is used to carry out electrolysis or hydrolysis, producing an acidic solution and an alkaline solution, the acidic solution being then used to decarbonate a starting metal carbonate, and the alkaline solution being then used to precipitate a metal hydroxide from the dissolved metal ions of the starting metal carbonate. In some embodiments, the volume concentrations of reactants on which such a reactor operates are determined by the pH values produced by the electrolyzer.

In accordance with certain embodiments, an alternative reactor concept is capable of producing higher concentrations of acid and base than the reactor in FIG. 3. In some embodiments, the system comprises a first reactor that electrolytically oxidizes a near-neutral solution of a dissolved metal salt to produce an alkaline solution, hydrogen, and a compound enriched in the anion of the metal salt. In some embodiments, the metal salt is an alkali halide salt or an alkaline earth halide salt, and said compound produced is a dihalide. A second reactor produces, in accordance with certain embodiments, an acidic solution by reacting said compound and hydrogen with water. Said acidic solution produced by the second reactor, and said alkaline solution produced by the first reactor, are then used, in some embodiments, to, respectively, dissolve said metal carbonate releasing CO₂, and precipitate said metal hydroxide. Unlike the reactor of FIG. 3, where reaching absolute H⁺ and OH⁻ concentrations greater than about 1 molar may be difficult, the alternative reactor can reach concentrations of 3 molar, 5 molar, or even higher, in certain embodiments.

In certain embodiments, the first reactor comprises a second electrode (e.g., the anode (e.g., 302)), a first electrode (e.g., the cathode (e.g., 301)), a semi-permeable membrane between the two electrodes, inlets for the electrolyte, and outlets for the products of electrolysis (H₂, a dihalide, and an alkaline solution). In some embodiments, an additional inlet in the vicinity of the first electrode introduces O₂. In some cases, the electrolyte is a near-neutral aqueous solution in which the metal salt is dissolved. In certain cases, the aqueous solution comprises halide anions (for example, F⁻, Cl⁻, Br⁻, I⁻) and the corresponding cations (for example, Li⁺, Na⁺, K⁺, NH₄⁺, Mg²⁺, Ca²⁺). In certain embodiments, the concentration of halide salt in the electrolyte may be anywhere from 0.01-50% by weight. In some embodiments, the electrolyte is introduced to the second electrode (e.g., the anode (e.g., 302)) by an inlet. In certain cases, the active material on the second electrode’s surface may comprise platinum, graphite, platinized titanium, mixed metal oxides, mixed metal oxide-clad titanium, platinized metal oxides (e.g. platinized lead oxide, manganese dioxide), platinized ferrosilicon, platinum-iridium alloys, ruthenium oxides, titanium oxides, ruthenium and/or titanium mixed metal oxides.

In some cases, at the second electrode (e.g., 302) in Reactor 1, halide anions are oxidized to produce dihalides (e.g. Cl₂. Br₂, I₂). For example, in certain instances, oxidation of dissolved Cl⁻ gives Cl₂ gas.

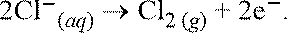

In certain embodiments, at room temperature, oxidation of Br⁻ gives Br₂, a fuming liquid, and oxidation of I⁻ gives I₂, a solid. The dihalide is collected from the electrolyzer, in some cases, through an outlet and is used to make acid in a subsequent step, described below. In certain instances, the electrolyte containing a cation (e.g. Li⁺, Na⁺, K⁺, NH₄⁺) moves through the semipermeable membrane (a diaphragm, or an ion-exchange membrane) towards the first electrode (e.g., cathode (e.g., 301)). In some cases, the diaphragm or membrane prevents the alkali solution generated at the first electrode from increasing the pH at the second electrode. In certain embodiments, the first electrode’s surface may comprise electrocatalytic compounds. Examples of electrocatalytic compounds include platinum, platinized titanium, mixed metal oxide-clad titanium, platinized metal oxides (e.g. platinized lead oxide, manganese dioxide), platinized ferrosilicon, platinum-iridium alloys, stainless steel, graphite, unalloyed titanium, stainless steel, nickel, nickel oxides. In certain embodiments, the second electrode comprises a metallic electrode, such as platinum, gold, nickel, iridium, copper, iron, steel, stainless steel, manganese, and zinc, or a carbon, such as graphite or disordered carbons, or a metal carbide, such as silicon carbide, titanium carbide, or tungsten carbide. In certain embodiments, the second electrode comprises a metal alloy (e.g. a nickel-chromium-iron alloy, nickel-molybdenum-cadmium alloy), a metal oxide (e.g. iridium oxide, nickel iron cobalt oxide, nickel cobalt oxide, lithium cobalt oxide, lanthanum strontium cobalt oxide, barium strontium ferrous oxide, manganese molybdenum oxide, ruthenium dioxide, iridium ruthenium tantalum oxide), a metal organic framework, or a metal sulfide (e.g. molybdenum sulfide). In certain embodiments, the electrocatalyst or electrode material is dispersed or coated onto a conductive support. In some embodiments, at the first electrode (e.g., the cathode (e.g., 301)) of Reactor 1, water is reduced to give OH⁻ (an alkali solution) and H₂_{(g):}

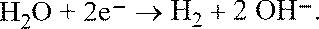

In another embodiment, at the first electrode (e.g., the cathode (e.g., 301)) of Reactor 1, O₂ is reduced to give OH⁻ (an alkali solution).

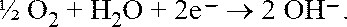

In some embodiments, the OH⁻ is charge-balanced by the cation in the electrolyte that crosses the diaphragm or membrane. In certain cases, the alkali hydroxide solution (e.g. NaOH, KOH), with a pH greater than 7, with a concentration of alkali 0.01 mol/L or more, is collected from the reactor from an outlet. In certain instances, the H₂ is collected from the reactor from a different outlet. In some cases, Reactor 1 produces an alkaline solution at one electrode, and hydrogen and a dihalide (in the instance where the metal salt is a metal halide) at the other electrode.

In accordance with some embodiments, Reactor 2 is a reactor that produces an acid by reacting the hydrogen gas and dihalide produced at the anode of Reactor 1, or by reacting the dihalide with water. Without being limited by the following examples, two embodiments of this reactor are as follows. In one embodiment, the reactor comprises a first chamber, an inlet through which H₂ is introduced to the first chamber, a second inlet through which the dihalide is introduced to the first chamber, and an outlet through which the hydrogen halide (e.g. HCl, HBr, HI) is removed from the first chamber, an inlet through which the hydrogen halide is introduced to a second chamber, an inlet through which water is introduced to a second chamber, and an outlet through which an aqueous, acidic solution of the hydrogen halide is removed from the second chamber. In some embodiments, in the first chamber the dihalide reacts with H₂ to form a hydrogen halide. In certain embodiments, the reaction between H₂ and the dihalide may be assisted by heating or irradiation by electromagnetic waves. For example, in some embodiments, if the dihalide is Cl₂, the following reaction takes place in Reactor 2:

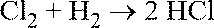

In some cases, in the second chamber, the hydrogen halide is dissolved in water to make an acidic solution. For example, HCl could be dissolved in water to make protons.

In accordance with another embodiment, the dihalide is reacted with water to produce the desired acid, and oxygen as a byproduct. In some cases, the exemplary reactor comprises a first chamber, an inlet through which H₂O is introduced to the first chamber, and a second inlet through which the dihalide is introduced to the first chamber. In certain instances, the reactor also comprises an outlet through which the hydrogen halide (e.g. HCl, HBr, HI) is removed from the first chamber, and an outlet through which O₂ is removed from the first chamber. In some cases, the reaction between chlorine as an exemplary dihalide and water is:

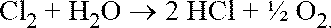

In some embodiments, the relative amounts of the dihalide and water will determine whether the pure hydrogen halide, or an admixture of the hydrogen halide and water, including for example a solution of the hydrogen halide in water, is produced. Optionally, in certain embodiments, the reactor may comprise a second chamber where the hydrogen halide is dissolved in water to make an acidic solution with an inlet through which the hydrogen halide is introduced, an inlet through which water is introduced to the second chamber, and an outlet through which an aqueous, acidic solution of the hydrogen halide is removed from the reactor.

In some embodiments, the system comprises an apparatus. In certain instances, the apparatus is a container (e.g., a container that is not open to the atmosphere). In accordance with certain embodiments, the apparatus is configured to collect one or more products or byproducts of the reactor (e.g., acid, base, hydrogen gas, oxygen gas, and/or carbon dioxide gas, etc.), store one or more of the one or more products or byproducts, and/or react one or more of the one or more products or byproducts (e.g., in a chemical dissolution and/or precipitation reaction).

In certain embodiments, the system comprises multiple apparatuses. Each apparatus may independently have one or more functions. Any apparatus, or configuration of apparatuses, disclosed herein may be used with any system disclosed herein. In certain embodiments, the apparatus is fluidically connected to the reactor. For example, in some instances, the apparatus is connected to the reactor by a conduit (e.g., a pipe, channel, needle, or tube) through which fluid can flow. In certain cases, an apparatus is fluidically connected to one or more other apparatuses (e.g., by a conduit, such as a pipe, channel, needle, or tube).

According to some embodiments, the method comprises collecting the acid and/or base. For example, in some embodiments, the method comprises removing the acid and/or base from the vessel in which it was produced (e.g., the reactor). A non-limiting example of a suitable method of collecting the acid and/or base comprises moving the acid and/or base through a conduit (e.g., a pipe, channel, needle, or tube) into a separate container. Other suitable examples of collecting the acid and/or base include moving the acid and/or base directly into a separate container (e.g., a container connected to the reactor by a panel that can be moved to block or allow diffusion of fluids). In some embodiments, the acid and/or base is collected continuously or in batches. In certain embodiments, the acid and/or base is collected automatically or manually.

In some embodiments, an apparatus is configured to collect an acid near the second electrode (and/or second reactor) and/or a base near the first electrode (and/or first reactor) (e.g., collect an acid from the acidic region and/or collect a base from the alkaline region). For example, referring to FIG. 3, in some embodiments, the system comprises a first apparatus which is configured to collect a base near the first electrode (e.g., 301). In certain embodiments, a second apparatus is configured to collect an acid near the second electrode (e.g., 302)(and/or second reactor) and/or a base near the first electrode (and/or first reactor). In some embodiments where the first apparatus is configured to collect a base near the first electrode, the second apparatus is configured to collect an acid near the second electrode.

In certain embodiments, collecting the acid comprises collecting acid produced by an electrode from a vicinity close enough to the electrode that the acid has not been significantly diluted and/or reacted (e.g., the pH of the collected acid is within 1 pH unit of the acid with the lowest pH in the reactor). Similarly, in some embodiments, collecting the base comprises collecting the base produced by the electrode from a vicinity close enough to the electrode that the base has not been significantly diluted and/or reacted (e.g., the pH of the collected base is within 1 pH unit of the base with the highest pH in the reactor).

According to some embodiments, the method comprises storing the acid and/or base. For example, in certain embodiments, once the acid and/or base are collected in a separate container, the method comprises keeping the acid and/or base in the separate container for at least some period of time. In some embodiments, the method comprises storing the acid and/or base for greater than or equal to 5 minutes, greater than or equal to 15 minutes, greater than or equal to 30 minutes, greater than or equal to 1 hour, greater than or equal to 5 hours, greater than or equal to 12 hours, greater than or equal to 1 day, greater than or equal to 2 days, greater than or equal to 3 days, greater than or equal to 1 week, greater than or equal to 2 weeks, or greater than or equal to 1 month. In certain embodiments, the method comprises storing the acid and/or base for less than or equal to 1 year, less than or equal to 6 months, less than or equal to 3 months, less than or equal to 2 months, less than or equal to 1 month, less than or equal to 2 weeks, less than or equal to 1 week, less than or equal to 3 days, less than or equal to 2 days, less than or equal to 1 day, or less than or equal to 12 hours. Combinations of these ranges are also possible (e.g., greater than or equal to 5 minutes and less than or equal to 1 year, greater than or equal to 5 hours and less than or equal to 1 day, or greater than or equal to 1 week and less than or equal to 1 year).

In some embodiments, an apparatus (e.g., the first apparatus and/or the second apparatus) is configured to react the acid in a chemical dissolution and/or in a precipitation reaction. In certain embodiments, an apparatus (e.g., the first apparatus and/or the second apparatus) is configured to react the base in a chemical dissolution and/or in a precipitation reaction. In some embodiments where the first apparatus is configured to react a base (e.g., in a chemical dissolution and/or in a precipitation reaction), the second apparatus is configured to react an acid (e.g., in a chemical dissolution and/or in a precipitation reaction).

According to certain embodiments, an apparatus (e.g., first apparatus and/or second apparatus) may be configured to (i) collect an acid near the second electrode and/or a base near the first electrode; (ii) store the acid and/or base; and/or (iii) react the acid and/or base (e.g., in a chemical dissolution and/or in a precipitation reaction).

According to some embodiments, each apparatus may have only one function. For example, in certain embodiments, a first apparatus is configured to collect a base near the first electrode, a second apparatus is configured to collect an acid near the second electrode, and a third apparatus is configured to react the base and/or acid (e.g., in a chemical dissolution and/or in a precipitation reaction). As another non-limiting example, in some embodiments, a first apparatus is configured to collect a base near the first electrode and store the base; a second apparatus is configured to collect an acid near the second electrode, store the acid, and react the acid (e.g., in a chemical dissolution and/or in a precipitation reaction); and a third apparatus is configured to react the base (e.g., in a chemical dissolution and/or in a precipitation reaction).

In yet another example, in some embodiments, a first apparatus is configured to collect a base near the first electrode (e.g., 301), a second apparatus is configured to collect an acid near the second electrode (e.g., 302), a third apparatus is configured to store the base, a fourth apparatus is configured to store the acid, a fifth apparatus is configured to react the base (e.g., in a chemical dissolution and/or in a precipitation reaction), and a sixth apparatus is configured to react the acid (e.g., in a chemical dissolution and/or in a precipitation reaction).

In certain embodiments, the reactor is intermittently run when in the first mode (e.g., as described above). In some cases, the reactor is continuously run in the first mode. In certain instances, the reactor is run intermittently in a first mode, while the reactions with the collected acid and or base (e.g., the chemical dissolution and/or precipitation reaction) are run continuously. For example, in some embodiments, the reactor produces enough acid and/or base when run in the first mode that it only needs to be run intermittently to produce enough acid and/or base to continuously perform the reactions (e.g., the chemical dissolution and/or precipitation reaction).

In some embodiments, a desired chemical reaction is conducted by collecting solutions or suspensions of differing compositions produced electrolytically, and using said solution or solutions to produce a product from said reactant in a portion of the reactor or in a separate apparatus. In accordance with some embodiments, an acidic solution is used to dissolve CaCO₃ in a first chamber, releasing CO₂ gas in the process. In a second chamber, in some embodiments, the dissolved solution reacts with the alkaline solution produced by the electrolyzer to produce Ca(OH)₂. In some embodiments, the two chambers are storage tanks for acidic and for alkaline solutions. In certain embodiments, the acid storage tank comprises a polymer material, or a glass lining. In some embodiments, the alkaline storage tank comprises a polymer material, or a metal. In some embodiments, the metal tank comprises iron or steel.

In certain cases, a byproduct of the precipitation reaction is fed back into the system (e.g., first reactor). In some instances, the system is configured to feed a byproduct from the precipitation reaction into the system (e.g., first reactor). In some embodiments, the byproduct has a neutral pH. For example, in certain cases, the byproduct has a pH of greater than 6, greater than or equal to 6.25, greater than or equal to 6.5, greater than or equal to 6.75, or greater than or equal to 6.9. In some instances, the byproduct has a pH of less than 8, less than or equal to 7.75, less than or equal to 7.5, less than or equal to 7.25, or less than or equal to 7.1. Combinations of these ranges are also possible (e.g., greater than 6 and less than 8 or greater than or equal to 6.9 and less than or equal to 7.1). In some embodiments, the byproduct has a pH of 7.

In some instances, the byproduct comprises an alkali halide (e.g., the byproduct in the precipitation of an alkali hydroxide) (e.g., NaCl). In certain cases, the byproduct comprises an alkali salt (e.g., NaClO₄, NaNO₃, sodium triflate, and/or sodium acetate).

In some embodiments, the acidic and/or basic solutions produced by the electrolysis reactor are at least partially collected and/or stored during periods of high electricity availability and/or low electricity cost, permitting the chemical dissolution reaction in the acid producing CO₂ and the chemical precipitation reaction occurring in the base to be conducted during periods of reduced or low electrolyzer operation or electricity availability and/or high electricity cost. In some embodiments, the storage of acidic and basic solutions functions as chemical storage, allowing the output of the chemically reacted product, which may generally be solid, liquid or gaseous, to be less variable, or to be smoothed, compared to the output rate of the electrolyzer. In some embodiments, the stored acidic or basic solutions are of a size or volume permitting the chemically reacted product to be produced at a rate that does not fully deplete the stored acidic or basic solutions during periods of reduced or low electrolyzer operation or electricity availability and/or high electricity cost. In some embodiments, a system comprises a source of variable electricity, said electrolyzer, and said chemical storage tanks and chemical reactor. In some embodiments, a method comprises operating such a system so as to produce a less variable, or constant or relatively constant, flow of a chemical reaction product from a more variable or intermittent electricity source.

In certain embodiments, the method comprises producing acid and base in a low-voltage mode (e.g., at a lower voltage than a high-voltage mode described herein). Any embodiment related to the low voltage mode may be used with any system disclosed herein. In some embodiments, the method does not produce oxygen gas and/or hydrogen gas. For example, in certain embodiments, the electrolytic reactions occurring in the low-voltage mode may be the oxidation of hydrogen at the second electrode (H₂ ➔ 2H⁺ + 2e⁻) and the reduction of water at the first electrode (2H₂O + 2e⁻ ➔ H₂ + 2OH⁻), such that oxygen gas is not produced. In another example, in certain embodiments, the electrolytic reactions occurring in the low-voltage mode may be the oxidation of water at the first electrode (2H₂O ➔ O₂ + 4H⁺ + 4e⁻) and the reduction of oxygen at the second electrode (O₂ + 2H₂O + 4e⁻ ➔ 4OH⁻), such that hydrogen gas is not produced.

In some embodiments, the reactor (e.g., 102, 202, 300) and/or system (e.g., system 100, 150, 200, etc.) that produces CO₂ also produces calcium hydroxide, also known as slaked lime, which can be decomposed to produce calcium oxide, also known as lime. In some embodiments, said lime or slaked lime is used in applications including but not limited to paper making, flue gas treatment carbon capture, plaster mixes and masonry (including Pozzolan cement), soil stabilization, pH adjustment, water treatment, waste treatment, and sugar refining. The following are non-limiting examples of the use of calcium hydroxide and/or calcium oxide which may be generated by systems of the various embodiments, such as system 100, 150, 200, etc.

### Metallurgical Uses

### Ferrous Metals

In some embodiments, slaked lime and/or lime produced by the reactors, systems, and/or methods disclosed herein is used in the making of iron and/or steel. For example, in the making of iron and/or steel, lime can be used as a flux, to form slag that prevents the iron and/or steel from oxidizing, and to remove impurities such as silica, phosphates, manganese and sulfur. In some cases, slaked lime (dry, or as a slurry) is used in the making of iron and/or steel as a lubricant for drawing wires or rods through dies, as a coating on casting molds to prevent sticking, and/or as a coating on steel products to prevent corrosion. In some instances, lime or slaked lime is also used to neutralize acidic wastes.

### Non-ferrous Metals

In some embodiments, slaked lime and/or lime produced by the reactors, systems, and/or methods disclosed herein is used in the making of nonferrous metals including, but not limited to, copper, mercury, silver, gold, zinc, nickel, lead, aluminum, uranium, magnesium and/or calcium. Lime may be used, in some cases, as a fluxing agent, to remove impurities (such as silica, alumina, phosphates, carbonates, sulfur, sulfates) from ores. For example, lime and slaked lime can be used in the flotation or recovery of non-ferrous ores. In certain cases, lime acts as a settling aid, to maintain proper alkalinity, and/or to remove impurities (such as sulfur and/or silicon). In some instances, in the smelting and refining of copper, zinc, lead and/or other non-ferrous ores, slaked lime is used to neutralize sulfurous gases and/or to prevent the formation of sulfuric acid. In certain instances, lime and/or slaked lime is also used as a coating on metals to prevent the reaction with sulfurous species. In certain cases, in the production of aluminum, lime and/or slaked lime is used to remove impurities (such as silica and/or carbonate) from bauxite ore, and/or is used to regulate pH. In some instances, lime is used to maintain alkaline pH for the dissolution of gold, silver, and/or nickel in cyanide extraction. In the production of zinc, lime is used as a reducing agent in certain cases. In some cases, in the production of metallic calcium and/or magnesium, magnesium and/or calcium oxides are reduced at high temperatures to form magnesium and/or calcium metal.

### Construction

### Masonry (other Than Portland Cement)

In some embodiments, slaked lime and/or lime produced by the reactors, systems, and/or methods disclosed herein is used for making masonry mortars, plasters, stuccos, whitewashes, grouts, bricks, boards, and/or non-Portland cements. In these applications, in certain embodiments, lime and/or slaked lime may be mixed with other additives and exposed to carbon dioxide to produce calcium carbonate, lime and/or slaked lime may be reacted with other additives (such as aluminosilicates) to form a cementitious material, and/or lime and/or slaked lime may be used as a source of calcium. In the instance of mortars, plasters, stuccos and whitewashes, in some cases, lime and/or slaked lime is mixed with additives and/or aggregates (such as sand) to form a paste/slurry that hardens as water evaporates and as the lime and/or slaked lime reacts with atmospheric carbon dioxide to form calcium carbonate. In the case of hydraulic pozzolan cements, in certain cases, lime and/or slaked lime is reacted with aluminates, silicates, and/or other pozzolanic materials (e.g., pulverized fuel ash, volcanic ash, blast furnace slag, and/or calcined clay), to form a water-based paste/slurry that hardens as insoluble calcium aluminosilicates are formed. In the case of other hydraulic cements, in some instances, lime and/or slaked lime is reacted at high temperature with sources of silica, alumina, and/or other additives such that cementitious compounds are formed, including dicalcium silicate, calcium aluminates, tricalcium silicate, and/or mono calcium silicate. In some cases, sandlime bricks are made by reacting slaked lime with a source of silica (e.g., sand, crushed siliceous stone, and/or flint) and/or other additives at temperatures required to form calcium silicates and/or calcium silicate hydrates. In some cases, lightweight concrete (e.g., aircrete) is made by reacting lime and/or slaked lime with reactive silica, aluminum powder, water, and/or other additives; the reaction between slaked lime and silicates/aluminates causes calcium silicates/aluminates and/or calcium silicate hydrates to form, while the reaction between water, slaked lime and aluminum causes hydrogen bubbles to form within the hardening paste. Whitewash is a white coating made from a suspension of slaked lime, which hardens and sets as slaked lime reacts with carbon dioxide from the atmosphere. Calcium silicate boards, concrete, and other cast calcium silicate products are formed, in some cases, when calcium silicate-forming materials (e.g., lime, slaked lime, silica, and/or cement) and additives (e.g., cellulose fiber and/or fire retardants) and water are mixed together, cast or pressed into shape. In some cases, high temperatures are used to react the lime, slaked lime, and/or silica, and/or to hydrate the cement.

### Soil Stabilization

In some embodiments, slaked lime and/or lime produced by the reactors, systems, and/or methods disclosed herein is used to stabilize, harden, and/or dry soils. For example, lime and/or slaked lime may be applied to loose or fine-grained soils before the construction of roads, runways, and/or railway tracks, and/or to stabilize embankments and/or slopes. In some cases, when lime is applied to clay soils a pozzolanic reaction may occur between the clay and the lime to produce calcium silicate hydrates, and/or calcium aluminate hydrates, which strengthen and/or harden the soil. In certain instances, lime and/or slaked lime applied to soils may also react with carbon dioxide to produce solid calcium carbonate, which may also strengthen and/or harden soil. In some cases, lime may also be used to dry wet soils at construction sites, as lime reacts readily with water to form slaked lime.

### Asphalt Additive and Asphalt Recycling

In some embodiments, slaked lime and/or lime produced by the reactors, systems, and/or methods disclosed herein is used to make and/or recycle asphalt. For example, in some cases, slaked lime is added to hot mix asphalt as a mineral filler and/or antioxidant, and/or to increase resistance to water stripping. In certain instances, slaked lime can react with aluminosilicates and/or carbon dioxide to create a solid product that improves the bond between the binder and aggregate in asphalt. As a mineral filler, in some instances, lime may increase the viscosity of the binder, the stiffness of the asphalt, the tensile strength of the asphalt, and/or the compressive strength of the asphalt. As a hydraulic road binder, in certain cases, lime may reduce moisture sensitivity and/or stripping, stiffen the binder so that it resists rutting, and/or improve toughness and/or resistance to fracture at low temperature. In some instances, lime and/or slaked lime added to recycled asphalt results in greater early strength and/or resistance to moisture damage.

### Waste Treatment, Water Treatment, Gas Treatment

### Gas Treatment

In some embodiments, slaked lime and/or lime produced by the reactors, systems, and/or methods disclosed herein is used for the removal of acid gases (such as hydrogen chloride, sulfur dioxide, sulfur trioxide, and/or hydrogen fluoride) and/or carbon dioxide from a gas mixture (e.g. flue gas, atmospheric air, air in storage rooms, and/or air in closed breathing environments such as submarines). For example, in some cases, lime and/or slaked lime is exposed to flue gas, causing the reaction of lime and/or slaked lime with components of the flue gas (such as acid gases, including hydrogen chloride, sulfur dioxide and/or carbon dioxide), resulting in the formation of non-gaseous calcium compounds (such as calcium chloride, calcium sulfite, and/or calcium carbonate). In certain embodiments, exposure of gas to slaked lime is done by spraying slaked lime solutions and/or slurries onto gas, and/or by reacting gas streams with dry lime and/or slaked lime. In certain embodiments, the gas stream containing acid gas or gases is first reacted with a solution of alkali metal hydroxides (e.g. sodium hydroxide and/or potassium hydroxide), to form a soluble intermediate species (such as potassium carbonate), which is subsequently reacted with lime and/or slaked lime to produce a solid calcium species (such as calcium carbonate) and regenerate the original alkali metal hydroxide solution. In some embodiments, the calcium carbonate formed from the reaction of lime and/or slaked lime with carbon dioxide or alkali carbonate is returned to the reactors, systems, and/or methods disclosed herein, so that the lime and/or slaked lime can be regenerated and/or so that the carbon dioxide can be sequestered.

### Non-Gaseous Waste Treatment

In some embodiments, slaked lime and/or lime produced by the reactors, systems, and/or methods disclosed herein is used to treat wastes such as biological wastes, industrial wastes, wastewaters, and/or sludges. In some cases, lime and/or slaked lime may be applied to the waste to create an alkaline environment, which serves to neutralize acid waste, inhibit pathogens, deter flies or rodents, control odors, prevent leaching, and/or stabilize and/or precipitate pollutants (such as heavy metals, chrome, copper, and/or suspended/dissolved solids) and/or dissolved ions that cause scaling (calcium and/or magnesium ions). In certain instances, lime may be used to de-water oily wastes. In some cases, slaked lime may be used to precipitate certain species, such as phosphates, nitrates, and/or sulfurous compounds, and/or prevent leaching. In certain instances, lime and/or slaked lime may be used to hasten the decomposition of organic matter, by maintaining alkaline conditions that favor hydrolysis.

### Water Treatment

In some embodiments, slaked lime and/or lime produced by the reactors, systems, and/or methods disclosed herein is used to treat water. For instance, lime and/or slaked lime may be used, in some cases, to create an alkaline environment, which serves to disinfect, remove suspended/colloidal material, reduce hardness, adjust pH, precipitate ions contributing to water hardness, precipitate dissolved metals (such as iron, aluminum, manganese, barium, cadmium, chromium, lead, copper, and/or nickel), and/or precipitate other ions (such as fluoride, sulfate, sulfite, phosphate, and/or nitrate).

### Agriculture and Food

### Agriculture

In some embodiments, slaked lime and/or lime produced by the reactors, systems, and/or methods disclosed herein is used for agriculture. For example, lime and/or slaked lime may be used alone, or as an additive in fertilizer, to adjust the pH of the soil and/or of the fertilizer mixture to give optimum growing conditions and/or improve crop yield, in some cases.

### Sugar

In some embodiments, slaked lime and/or lime produced by the reactors, systems, and/or methods disclosed herein is used to refine sugar. For example, in some cases, lime and/or slaked lime is used to raise the pH of raw sugar juice, destroy enzymes in the raw sugar juice, and/or react with inorganic and/or organic species to form precipitates. Excess calcium may be precipitated with carbon dioxide, in certain instances. In certain cases, the precipitated calcium carbonate that results may be returned to the reactors, systems, and/or methods disclosed herein, to regenerate slaked lime.

### Leather

In some embodiments, slaked lime and/or lime produced by the reactors, systems, and/or methods disclosed herein is used to make leather and/or parchment. In the leather making process, lime is used, in some cases, to remove hair and/or keratin from hides, split fibers, and/or remove fat.

### Glue, Gelatin

In some embodiments, slaked lime and/or lime produced by the reactors, systems, and/or methods disclosed herein is used to make glue and/or gelatin. In the process of making glue and/or gelatin, in some cases, animal bones and/or hides are soaked in slaked lime, causing collagen and other proteins to hydrolyze, forming a mixture of protein fragments of different molecular weights.

### Dairy Products

In some embodiments, slaked lime and/or lime produced by the reactors, systems, and/or methods disclosed herein is used to make dairy products. In some cases, slaked lime is used to neutralize acidity of cream before pasteurization. In certain cases, slaked lime is used to precipitate calcium caseinate from acidic solutions of casein. In some instances, slaked lime is added to fermented skim milk to produce calcium lactate.

### Fruit Industry

In some embodiments, slaked lime and/or lime produced by the reactors, systems, and/or methods disclosed herein is used in the fruit industry. For example, slaked lime and/or lime is used, in some cases, to remove carbon dioxide from air in fruit storage. In some instances, slaked lime is used to neutralize waste citric acid and to raise the pH of fruit juices.

### Insecticides/Fungicides

In some embodiments, slaked lime and/or lime produced by the reactors, systems, and/or methods disclosed herein is used as an additive in fungicides and/or insecticides. For example, slaked lime may be mixed with coper sulfate to form tetracupric sulfate, a pesticide. In some cases, lime may also be used as a carrier for other kinds of pesticides, as it forms a film on foliage as it carbonates, retaining the insecticide on the leaves. In some instances, slaked lime is used to control infestations of starfish on oyster beds.

### Food Additive

In some embodiments, slaked lime and/or lime produced by the reactors, systems, and/or methods disclosed herein is used as a food additive. In some cases, lime and/or slaked lime may be used as an acidity regulator, as a pickling agent, to remove cellulose (e.g. from kernels such as maize), and/or to precipitate certain anions (such as carbonates) from brines.

### Chemicals

In some embodiments, slaked lime and/or lime produced by the reactors, systems, and/or methods disclosed herein is used to make chemicals. For example, lime and/or slaked lime may be used as a source of calcium and/or magnesium, an alkali, a desiccant, causticizing agent, saponifying agent, bonding agent, flocculant and/or precipitant, fluxing agent, glass-forming product, degrader of organic matter, lubricant, filler, and/or hydrolyzing agent, among other things.

### Inorganic Calcium Compounds

### Precipitated Calcium Carbonate

In some embodiments, slaked lime and/or lime produced by the reactors, systems, and/or methods disclosed herein is used to make precipitated calcium carbonate. In some instances, a solution and/or slurry of slaked lime, and/or a solution of calcium ions, is reacted with carbon dioxide, and/or an alkali carbonate, so that a precipitate of calcium carbonate and/or magnesium carbonate forms. In certain instances, the precipitated alkali metal carbonate may be used as a filler, to reduce shrinkage, improve adhesion, increase density, modify rheology and/or to whiten/brighten plastics (such as PVC and latex), rubber, paper, paints, inks, cosmetics, and/or other coatings. Precipitated carbonates, in some cases, may be used as flame retarders or dusting powder. In certain cases, precipitated calcium carbonate may be used as an alkalizer, for agriculture, as an antiseptic agent, flour additive, brewing additive, digestive aid, and/or additive for bituminous products), an abrasive (in cleaners, detergents, polishes and/or toothpastes), a dispersant in pesticides, and/or a desiccant.

### Calcium Hypochlorite

In some embodiments, slaked lime and/or lime produced by the reactors, systems, and/or methods disclosed herein is used to make calcium hypochlorite, a bleach, by reacting chlorine with lime and/or slaked lime.

### Calcium Carbide

In some embodiments, slaked lime and/or lime produced by the reactors, systems, and/or methods disclosed herein is used to make calcium carbide, a precursor to acetylene, by reacting lime with carbonaceous matter (e.g. coke) at high temperature.

### Calcium Phosphates

In some embodiments, slaked lime and/or lime produced by the reactors, systems, and/or methods disclosed herein is used to make calcium phosphates (monocalcium phosphate, dicalcium phosphate, and/or tricalcium phosphate) by reacting phosphoric acid with slaked lime, and/or aqueous calcium ions, in the appropriate ratios. In some cases, monocalcium phosphate may be used as an additive in self-rising flour, mineral enrichment foods, as a stabilizer for milk products and/or as a feedstuff additive. In some instances, dicalcium phosphate dihydrate is used in toothpastes, as a mild abrasive, for mineral enrichment of foodstuffs, as a pelletizing aid and/or as a thickening agent. In certain instances, tricalcium phosphate is used in toothpastes, and/or as an anti-caking agent in foodstuffs and/or fertilizers.

### Calcium Bromide

In some embodiments, slaked lime and/or lime produced by the reactors, systems, and/or methods disclosed herein is used to make calcium bromide. This is done, in some cases, by reacting lime and/or slaked lime with hydrobromic acid and/or bromine and a reducing agent (e.g. formic acid and/or formaldehyde).

### Calcium Hexacyanoferrate

In some embodiments, slaked lime and/or lime produced by the reactors, systems, and/or methods disclosed herein is used to make calcium hexacyanoferrate, by reacting lime and/or slaked lime with hydrogen cyanide in an aqueous solution of ferrous chloride. Calcium hexacyanoferrate can then be converted to the alkali metal salt, or hexacyanoferrates. These are used as pigments and anti-caking agents.

### Calcium Silicon

In some embodiments, slaked lime and/or lime produced by the reactors, systems, and/or methods disclosed herein is used to make calcium silicon, by reacting lime, quartz and/or carbonaceous material at high temperatures. In some cases, calcium silicon is used as a de-oxidizer, as a de-sulfurizer, and/or to modify non-metallic inclusions in ferrous metals.

### Calcium Dichromate

In some embodiments, slaked lime and/or lime produced by the reactors, systems, and/or methods disclosed herein is used to make calcium dichromate, by roasting chromate ores with lime.

### Calcium Tungstate

In some embodiments, slaked lime and/or lime produced by the reactors, systems, and/or methods disclosed herein is used to make calcium tungstate, by reacting lime and/or slaked lime with sodium tungstate, to be used in the production of ferrotungsten and/or phosphors for items such as lasers, fluorescent lamps and/or oscilloscopes.

### Organic Calcium Compounds

### Calcium Citrate

In some embodiments, slaked lime and/or lime produced by the reactors, systems, and/or methods disclosed herein is used to make calcium citrate, by reacting lime and/or slaked lime with citric acid. In some cases, the calcium citrate may be reacted with sulfuric acid to regenerate pure citric acid.

### Calcium Soaps

In some embodiments, slaked lime and/or lime produced by the reactors, systems, and/or methods disclosed herein is used to make calcium soaps, by reacting slaked lime with aliphatic acids, wax acids, unsaturated carboxylic acids (e.g. oleic acid, linoleic acid, ethylhexanoate acids), napthenic acids, and/or resin acids. In some cases, calcium soaps are used as lubricants, stabilizers, mold-release agents, waterproofing agents, coatings, and/or additives in printing inks.

### Calcium Lactate

In some embodiments, slaked lime and/or lime produced by the reactors, systems, and/or methods disclosed herein is used to make calcium lactate, by reacting slaked lime with lactic acid. In certain instances, the lactic acid may be reacted in a second step with sulfuric acid to produce pure lactic acid. In some instances, these chemicals act as coagulants and foaming agents. In some cases, calcium lactate is used as a source of calcium in pharmaceutical agents and/or foodstuffs, and/or as a buffer.

### Calcium Tartarate

In some embodiments, slaked lime and/or lime produced by the reactors, systems, and/or methods disclosed herein is used to make calcium tartrate, by reacting slaked lime with alkali bitartarates. In some cases, the calcium bitartarate may be reacted in a second step with sulfuric acid to produce pure tartaric acid. In certain instances, tartaric acid is used in foodstuffs, pharmaceutical preparations, and/or as an additive in plaster and/or metal polish.

### Inorganic Chemicals

### Aluminum Oxide

In some embodiments, slaked lime and/or lime produced by the reactors, systems, and/or methods disclosed herein is used to make aluminum oxide. Lime is used to precipitate impurities (e.g., silicates, carbonates, and/or phosphates) from processed bauxite ore in the preparation of aluminum oxide.

### Alkali Carbonates and Bicarbonates

In some embodiments, slaked lime and/or lime produced by the reactors, systems, and/or methods disclosed herein is used to make alkali carbonates and/or bicarbonates from alkali chlorides in the ammonia-soda process. In this process, in some cases, lime and/or slaked lime is reacted with ammonium chloride (and/or ammonium chlorides, such as isopropylammonium chloride) to regenerate ammonia (and/or amines, such as isopropyl amine) after the reaction of ammonia (and/or the amine) with an alkali chloride. In some cases, the resulting calcium chloride can be reacted with the alkaline stream from the reactors, systems, and/or methods disclosed herein, to regenerate the slaked lime.

### Strontium Carbonate

In some embodiments, slaked lime and/or lime produced by the reactors, systems, and/or methods disclosed herein is used to make strontium carbonate. In some instances, lime and/or slaked lime is used to re-generate ammonia from ammonium sulfate, which forms after the ammonia has been carbonated and reacted with strontium sulfate.

### Calcium Zirconate

In some embodiments, slaked lime and/or lime produced by the reactors, systems, and/or methods disclosed herein is used to make calcium zirconate. In some cases, lime and/or slaked lime reacts with zircon, ZrSiO₄, to produce a calcium silicate and zirconate, which is further purified.

### Alkali Hydroxides

In some embodiments, slaked lime and/or lime produced by the reactors, systems, and/or methods disclosed herein is used to make alkali hydroxides from alkali carbonates, in a process often called causticizing or re-causticizing. In some cases, slaked lime is reacted with alkali carbonates to produce alkali hydroxides and calcium carbonate. The process of causticizing alkali carbonates is a feature of several other processes, in some instances, including the purification of bauxite ore, the processing of carbolic oil, and the Kraft liquor cycle (in which “green liquor”, containing sodium carbonate, reacts with slaked lime to form “white liquor”, containing sodium hydroxide).

### Magnesium Hydroxide

In some embodiments, slaked lime and/or lime produced by the reactors, systems, and/or methods disclosed herein is used to make magnesium hydroxide. In some cases, the addition of slaked lime to solutions containing magnesium ions (e.g. seawater and/or brine solutions) causes magnesium hydroxide to precipitate from solution.

### Organic Chemicals

### Alkene Oxides.

In some embodiments, slaked lime and/or lime produced by the reactors, systems, and/or methods disclosed herein is used to make alkene oxides. In some instances, lime is used to saponify or dehydrochlorinate propylene and/or butene chlorohydrins to produce the corresponding oxides. The oxides may then be converted to the glycols by acidic hydrolysis, in some instances.

### Diacetone Alcohol.

In some embodiments, slaked lime and/or lime produced by the reactors, systems, and/or methods disclosed herein is used to make diacetone alcohol. In some cases, slaked lime is used as an alkaline catalyst to promote the self-condensation of acetone to form diacetone alcohol, which is used as a solvent for resins, and/or as in intermediate in the production of mesityl oxide, methyl isobutyl ketone and/or hexylene glycol.

### Hydroxypivalic Acid Neopentyl Glycol Ester, Pentaerythritol.

In some embodiments, slaked lime and/or lime produced by the reactors, systems, and/or methods disclosed herein is used as a basic catalyst to make hydroxypivalic acid neopentyl glycol ester, and/or pentaerythritol.

### Anthraquinone Dyes and Intermediates.

In some embodiments, slaked lime and/or lime produced by the reactors, systems, and/or methods disclosed herein is used as a basic reagent, to replace a sulfonic acid group with a hydroxide, in the making of anthraquinone dyes and/or intermediates.

### Trichloroethylene

In some embodiments, slaked lime and/or lime produced by the reactors, systems, and/or methods disclosed herein is used to remove a chlorine from tetrachloroethane to form trichloroethylene.

### Miscellaneous Uses

### Silica, Silicon Carbide and Zirconia Refractories.

In some embodiments, slaked lime and/or lime produced by the reactors, systems, and/or methods disclosed herein is used as a binder, bonding and/or stabilizing agent in the fabrication of silica, silicon carbide and/or zirconia refractories.

### Lime Glass.

In some embodiments, slaked lime and/or lime produced by the reactors, systems, and/or methods disclosed herein is used as a source of lime in the fabrication of soda-lime glass. In some instances, lime and/or slaked lime is heated to high temperatures with other raw materials, including silica, sodium carbonate and/or additives such as alumina and/or magnesium oxide. In some instances, the molten mixture forms a glass upon cooling.

### Whiteware Pottery and Vitreous Enamels.

In some embodiments, slaked lime and/or lime produced by the reactors, systems, and/or methods disclosed herein is used to make whiteware pottery and/or vitreous enamels. In certain cases, slaked lime is blended with clays to act as a flux, a glass-former, to help bind the materials, and/or to increase the whiteness of the final product.

### Lubricant for Casting and Drawing.

In some embodiments, slaked lime and/or lime produced by the reactors, systems, and/or methods disclosed herein is used as a lubricant for casting and/or drawing of materials (such as iron, aluminum, copper, steel and/or noble metals). In some instances, calcium-based lubricants can be used at high temperature to prevent the metal from sticking to the mold. In certain cases, lubricants can be calcium soaps, blends of lime and other materials (including silicilic acid, aluminia, carbon and/or fluxing agents such as fluorospar and/or alkali oxides). Slaked lime is used as a lubricant carrier, in some cases. In certain instances, the slaked lime bonds to the surface of the wire, increases surface roughness and/or improves adhesion of the drawing compound.

### Drilling Muds.

In some embodiments, slaked lime and/or lime produced by the reactors, systems, and/or methods disclosed herein is used in drilling mud formulations to maintain high alkalinity and/or to keep clay in a non-plastic state. Drilling mud may, in some cases, be pumped through a hollow drill tube when drilling through rock for oil and gas. In certain instances, the drilling mud carries fragments of rock produced by the drill bit to the surface.

### Oil Additives and Lubricating Greases.

In some embodiments, slaked lime and/or lime produced by the reactors, systems, and/or methods disclosed herein is used as an oil additive and/or lubricating grease. In some instances, lime is reacted with alkyl phenates and/or organic sulfonates to make calcium soaps, which are blended with other additives to make oil additives and/or lubricating greases. In some cases, the lime-based additives prevent sludge build-up and to reduce acidity from products of combustion, especially at high temperature.

### Pulp and Paper

In some embodiments, slaked lime and/or lime produced by the reactors, systems, and/or methods disclosed herein is used in the pulp and/or paper industry. For example, slaked lime is used in the Kraft process to re-causticize the sodium carbonate into sodium hydroxide. In some cases, the calcium carbonate that forms from this reaction can be returned to the reactors, systems and/or methods disclosed herein to regenerate the slaked lime. In certain instances, slaked lime can also be used as a source of alkali in the sulfite process of pulping, to prepare the liquor. In certain cases, slaked lime is added to a solution of sulfurous acid to form a bisulfite salt. The mixture of sulfurous acid and bisulfite is used, in some cases, to digest the pulp. Slaked lime can also be used to precipitate calcium lignosulfonates from spent sulfite liquor, in certain instances.

### Aquariums

In some embodiments, slaked lime and/or lime produced by the reactors, systems, and/or methods disclosed herein is used as a source of calcium and/or alkalinity for marine aquariums and/or reef growth.

### A Method of Storing Heat

In some embodiments, slaked lime and/or lime produced by the reactors, systems, and/or methods disclosed herein is used for thermochemical energy storage (e.g. for a self-heating food container and/or for solar heat storage).

### Fire Retardant

In some embodiments, calcium and/or magnesium hydroxide produced by the reactors, systems, and/or methods disclosed herein is used as a fire retardant, an additive to cable insulation, and/or insulation of plastics.

### Antimicrobial Agent

In some embodiments, slaked lime and/or lime produced by the reactors, systems, and/or methods disclosed herein is used as an antimicrobial agent. For example, in some instances, lime and/or slaked lime is used to treat disease contaminated areas, such as walls, floors, bedding, and/or animal houses.

### Applications in Agriculture

### Electrochemically Generated Supplemental CO₂ to Increase Productivity of Indoor Farms and Greenhouses

The process of photosynthesis involves the fixation of inorganic carbon from atmosphere, using energy from the sun to convert CO₂ entering plants into organic carbons in the form of sugars. These sugars serve as food and as new material for plant growth, integral to the continued survival and development of plants. In greenhouses and indoor farms, CO₂ is often the limiting factor for plant growth; while typically sunlight, nutrients, and water are well-supplied, CO₂ levels are depleted, as plants take in much more CO₂ during the day than they produce through cellular respiration. Though CO₂ concentrations in normal air is approximately 400 parts per million, optimal levels for plant growth range from 600 to 1500 parts per million. Therefore, supplementing additional CO₂ to raise atmospheric levels above ambient concentrations in greenhouses and indoor farms is a well-recognized practice, resulting in an average 20% increase in crop yields, depending on the type of plant.

Of all of the various methods used by farmers to increase CO₂ levels-including providing compressed CO₂ gas from tanks, vaporizing liquid CO₂, or allowing dry ice to sublimate-the most common and largest scale practice is the combustion of hydrocarbon fuels, such as natural gas and propane, by a CO₂ generator or combustion engine. Though the cost of such an operation is subject to the price of the fuel, it is typically seen as the most inexpensive method to supplement CO₂, especially in large greenhouses which require greater quantities. In this method, the cost of CO₂ is directly tied to the cost of fuel, and may be about $100 per tonne of CO₂. Using a gas engine also produces heat, which may be used to raise the temperature of the indoor farm or greenhouse at night. However, burning fossil fuels is clearly not the most environmentally-friendly method of supplementing CO₂ if sources that are a byproduct of other industrial processes can instead be utilized, since supplemental CO₂ is not completely consumed and there is typically loss to the atmosphere. Additionally, incomplete combustion of fuels can produce in harmful impurities, such as nitrous oxides and ethylene, which can result in necrosis and other negative impacts that may lower the quality of or wipe out an entire crop.

Thus, there appears to be an opportunity in the agriculture industry for an alternative method of CO₂ supplementation, namely through the local provision of the CO₂ byproduct from embodiments of the invention. The pure, humid streams of CO₂ would inherently be free of the harmful impurities present in exhaust or flue gas derived from combustion of fossil fuels. In some embodiments, dehydration of the CO₂ stream produced according to the invention may be unnecessary, as the ideal humidity level for greenhouses is relatively high, at approximately 60%. Introducing the CO₂ produced by embodiments of the invention into greenhouses and indoor farms would not only allow CO₂ that would otherwise contribute to atmospheric levels to be converted to sugars to bolster plant growth, but it would also mitigate the agricultural demand for fossil fuels and the negative consequences of fuel combustion.

Various embodiments may provide a method comprising the use of an electrochemical reactor to produce at least an acid, the dissolution of a metal carbonate in said acid releasing CO₂, and the use of said CO₂ to improve the health or growth rate or yield of an organism. In some embodiments, the organism is a plant. In some embodiments, the organism is an animal. In some embodiments, the organism is photosynthetic bacteria. In some embodiments, the organism comprises an agricultural product. In some embodiments, said agricultural product comprises food for humans or animals. In some embodiments, the plant comprises algae. In some embodiments, the algae is used for food. In some embodiments, the algae is used to produce a biofuel. In some embodiments, the plant is used to produce a biofuel. In some embodiments, said electrochemical reactor is powered by electricity from a renewable resource. In some embodiments, said renewable resource is solar or wind energy. In some embodiments, said acid or said CO₂ is stored for later use.

Various embodiments may provide an apparatus, comprising an electrochemical reactor that produces at least an acid, a subreactor or other apparatus for the dissolution of a metal carbonate in said acid, and a means of collecting the CO₂ produced upon dissolution of said metal carbonate in said acid. In some embodiments, said subreactor is a region within said reactor. In some embodiments, said subreactor is a separate apparatus from said reactor. In some embodiments, the apparatus further comprises an apparatus for storing the acid, a means of compressing the CO₂, storing the CO₂.

Various embodiments may provide a system comprising a renewable electricity source, an electrochemical reactor, and a facility containing living organisms. In some embodiments, said facility is used for farming or biofuel production. In some embodiments, said facility comprises an indoor farm, a greenhouse, or a biofuel production facility. In some embodiments, the electrochemical reactor is powered at least in part by said renewable electricity source, and produces at least an acid. In some embodiments, also comprising a means of dissolving a metal carbonate in said acid releasing CO₂. In some embodiments, also comprising a means for collecting said CO₂. In some embodiments, said CO₂ is supplied to said facility. In some embodiments, said organisms are plants or photosynthetic bacteria. In some embodiments, said facility is a medical care facility.

Various embodiments may provide a system comprising a renewable electricity source, an electrochemical reactor, and a facility, the system configured to produce CO₂ at least in part by the electrochemical reactor power being at least partially powered by the renewable electricity source and provide the CO₂ to the facility. In some embodiments, said CO₂ is used for various purposes as discussed herein.f

Various examples of aspects of the various embodiments are described in the following paragraphs.

Example 1. A method comprising the use of an electrochemical reactor to produce at least an acid, the dissolution of a metal carbonate in said acid releasing CO₂, and the use of said CO₂ to improve the health or growth rate or yield of an organism.

Example 2. The method of example 1, wherein the organism is a plant.

Example 3. The method of example 1, wherein the organism is an animal.

Example 4. The method of example 1, wherein the organism is photosynthetic bacteria.

Example 5. The method of example 1, wherein the organism comprises an agricultural product.

Example 6. The method of example 5, wherein said agricultural product comprises food for humans or animals.

Example 7. The method of example 2, wherein the plant comprises algae.

Example 8. The method of example 7, wherein the algae is used for food.

Example 9. The method of example 7, wherein the algae is used to produce a biofuel.

Example 10. The method of example 2, wherein the plant is used to produce a biofuel.

Example 11. The method of example 1, wherein said electrochemical reactor is powered by electricity from a renewable resource.

Example 12. The method of example 9, wherein said renewable resource is solar or wind energy.

Example 13. The method of example 1, wherein said acid or said CO₂ is stored for later use.

Example 14. An apparatus, comprising an electrochemical reactor that produces at least an acid, a subreactor or other apparatus for the dissolution of a metal carbonate in said acid, and a means of collecting the CO₂ produced upon dissolution of said metal carbonate in said acid.

Example 15. The apparatus of example 12 wherein said subreactor is a region within said reactor.

Example 16. The apparatus of example 12 wherein said subreactor is a separate apparatus from said reactor.

Example 17. The apparatus of examples 14-16, including an apparatus for storing the acid, a means of compressing the CO₂, storing the CO₂.

Example 18. A system comprising a renewable electricity source, an electrochemical reactor, and a facility containing living organisms.

Example 19. The system of example 18, wherein said facility is used for farming or biofuel production.

Example 20. The system of example 18, wherein said facility comprises an indoor farm, a greenhouse, or a biofuel production facility.

Example 21. The system of example 18, wherein the electrochemical reactor is powered at least in part by said renewable electricity source, and produces at least an acid.

Example 22. The system of example 18, also comprising a means of dissolving a metal carbonate in said acid releasing CO₂.

Example 23. The system of example 22, also comprising a means for collecting said CO₂.

Example 24. The system of example 22 or 23, wherein said CO₂ is supplied to said facility.

Example 25. The system of example 18, wherein said organisms are plants or photosynthetic bacteria.

Example 26. The system of example 18, wherein said facility is a medical care facility.

Example 27. A system comprising a renewable electricity source, an electrochemical reactor, and a facility.

Example 28. The system of any of examples 18-27, the system configured to produce CO₂ at least in part by the electrochemical reactor power being at least partially powered by the renewable electricity source and provide the CO₂ to the facility.

Example 29. The system of example 28, wherein said CO₂ is used for any purpose as discussed herein.

Example 30. A system, comprising: an electrochemical reactor configured to produce at least an acid; a second device configured to produce CO₂ at least in part from dissolution of a metal carbonate in said acid; and a collection device configured to collect the produced CO₂ produced upon dissolution of said metal carbonate in said acid and provide the produced CO₂ to a storage system and/or operating system, wherein the storage system and/or operating system are configured to enable the produced CO₂ to be applied to, or used by, another system for one or more purposes.

Example 31. The system of example 30, further comprising a renewable power source providing power to the electrochemical reactor.

Example 32. The system of any of examples 30-31, wherein the second device is included in the electrochemical reactor or is separate from the electrochemical reactor.

Example 33. The system of any of examples 30-32, wherein the reactor is a subreactor.

Example 34. The system of any of examples 30-33, wherein the metal carbonate is part of a material containing metal carbonate.

Example 35. The system of example 34, wherein the material containing metal carbonate is a natural material, synthesized material, or waste material as described herein.

Example 36. The system of any of examples 30-35, wherein the one or more purposes comprise agricultural or aquaculture purposes.

Example 37. The system of any of examples 30-35, wherein the one or more purposes comprise use as an inert gas for chemical processes, welding, as a lasing medium, preventing spoilage of foods and other air-sensitive materials, to extinguish fires, to dilute flammable or toxic vapours, as a non-reactive cooling gas; as a toxic gas to terminate or subdue animals, as a medical gas, as a propellant, as a food additive, as a reagent, as a component for the production of building materials, as pest control mechanism, as an algae growth promotor, as a coral growth promotor, as an oil recovery pressurizing and/or flow agent, as a cleaning agent, as a solvent, or as a refrigerant.

Example 38. A method comprising, operating a system according to any of examples 30-37.

Example 39. A method, system, or device as described herein.

Example 40. A method, comprising: electrochemically dissolving a metal carbonate to release CO₂; and using the released CO₂ to improve the health or growth rate or yield of an organism.

Example 41. The method of example 40, further comprising electrochemically generating an acid to dissolve the metal carbonate.

Example 42. The method of example 41, wherein electrochemically generating the acid comprising electrolyzing water to generate the acid which comprises hydrogen ions.

Example 43. The method of example 42, wherein the hydrogen ions react with the metal carbonate to generate metal ions and the released CO₂

Example 44. The method of example 43, further comprising electrochemically generating hydroxide ions and reacting the hydroxide ions with the metal ions to form a metal hydroxide solid.

Example 45. The method of example 44, wherein the metal carbonate comprises calcium carbonate and the metal hydroxide solid comprises calcium hydroxide solid.

Example 46. A system comprising: an electrochemical device configured to electrochemically dissolve a metal carbonate to release CO₂; and a collection device configured to collect the released CO₂ and provide the collected CO₂ to a storage system and/or operating system, wherein the storage system and/or operating system are configured to enable the collected CO₂ to be applied to, or used by, another system for one or more purposes.

Example 47. The system of example 46, wherein the electrochemical device comprises an electrolyzer which is configured to electrochemically generate an acid to dissolve the metal carbonate located in the electrolyzer.

Example 48. The system of example 47, wherein the electrolyzer comprises a water tank, two electrodes located in the water tank, and a current or voltage source, wherein the water tank is configured to hold water and the metal carbonate.

Example 49. A method, comprising: dissolving a metal carbonate using an acid to release CO₂; and using the released CO₂ to improve the health or growth rate or yield of an organism.

The foregoing method descriptions are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not necessarily intended to limit the order of the steps; these words may be used to guide the reader through the description of the methods. Further, any reference to claim elements in the singular, for example, using the articles “a,” “an” or “the” is not to be construed as limiting the element to the singular.

Further, any step of any embodiment described herein can be used in any other embodiment. The preceding description of the disclosed aspects is provided to enable any person skilled in the art to make or use the present invention. Various modifications to these aspects will be readily apparent to those skilled in the art, and the generic principles defined herein may be applied to other aspects without departing from the scope of the invention. Thus, the present invention is not intended to be limited to the aspects shown herein but is to be accorded the widest scope consistent with the principles and novel features disclosed herein.

What is claimed is:

1. A method, comprising:
using a reactor to produce at least an acid;
releasing CO₂ by dissolution of a metal carbonate in the acid; and
using the CO₂ to improve a health, a grow rate, and/or a yield of an organism.

2. The method of claim 1, wherein the organism is an animal.

3. The method of claim 1, wherein the organism is photosynthetic bacteria.

4. The method of claim 1, wherein the organism comprises an agricultural product.

5. The method of claim 4, wherein the agricultural product comprises food for humans or animals.

6. The method of claim 1, wherein the organism is a plant.

7. The method of claim 6, wherein the plant comprises algae.

8. The method of claim 7, further comprising using the algae for food.

9. The method of claim 7, further comprising using the algae to produce a biofuel.

10. The method of claim 6, further comprising using the plant to produce a biofuel.

11. The method of claim 1, wherein the reactor is an electrochemical reactor.

12. The method of claim 11, further comprising powering the electrochemical reactor by electricity from a renewable resource.

13. The method of claim 12, wherein the renewable resource is solar or wind energy.

14. The method of claim 1, further comprising storing the acid and/or the CO₂.

15. The method of claim 1, wherein the dissolution of the metal carbonate in the acid occurs under pressure greater than one atmosphere.

16. An apparatus, comprising:
a reactor that is configured to produce at least an acid;
a sub reactor configured for the dissolution of a metal carbonate in the acid; and
a means of collecting CO₂ produced upon dissolution of the metal carbonate in the acid.

17. The apparatus of claim 16, wherein the reactor is an electrochemical reactor.

18. The apparatus of claim 17 wherein the subreactor is a region within the reactor.

19. The apparatus of claim 17 wherein the subreactor is separated from the reactor.

20. The apparatus of claim 16, further comprising:
an apparatus for storing the acid;
a means for compressing the CO₂; and
a means for storing the CO₂.

21. The apparatus of claim 16, wherein the subreactor comprises a pressure vessel configured to operate such that the CO₂ is produced under a pressure greater than one atmosphere.

22. A system, comprising:
a renewable electricity source;
a reactor; and
a facility containing living organisms.

23. The system of claim 22, wherein the facility is used for farming or biofuel production.

24. The system of claim 22, wherein the facility comprises an indoor farm, a greenhouse, or a biofuel production facility.

25. The system of claim 22, wherein the reactor is powered at least in part by the renewable electricity source and the reactor produces an acid.

26. The system of claim 25, wherein the reactor is an electrochemical reactor.

27. The system of claim 25, further comprising a means of dissolving a metal carbonate in the acid thereby releasing CO₂.

28. The system of claim 27, further comprising a means for collecting the CO₂.

29. The system of claim 27, wherein the CO₂ is supplied to the facility.

30. The system of claim 27, wherein the means of dissolving the metal carbonate in the acid comprises a pressure vessel configured such that the released CO₂ is under a pressure greater than one atmosphere.

31. The system of claim 26, wherein the system is configured to produce CO₂ at least in part by the electrochemical reactor being at least partially powered by the renewable electricity source and the system is configured to provide the CO₂ to the facility.

32. The system of claim 22, wherein the organisms are plants or photosynthetic bacteria.

33. The system of claim 22, wherein the facility is a medical care facility.

34. A system, comprising:
a reactor configured to produce at least an acid;
a second device configured to produce CO₂ at least in part from dissolution of a metal carbonate in the acid; and
a collection device configured to:
collect the produced CO₂ produced upon dissolution of the metal carbonate in the acid; and
provide the produced CO₂ to a storage system and/or operating system, wherein the storage system and/or operating system is configured to enable the produced CO₂ to be applied to, or used by, another system.

35. The system of claim 34, wherein the reactor is an electrochemical reactor.

36. The system of claim 35, further comprising a renewable power source providing power to the electrochemical reactor.

37. The system of claim 35, wherein the second device is included in the electrochemical reactor or is separate from the electrochemical reactor.

38. The system of claim 35, wherein the second device comprises a pressure vessel pressurized such that the released CO₂ is released into the interior of the pressure vessel under a pressure greater than a pressure outside the pressure vessel.

39. The system of claim 34, wherein the reactor is a subreactor.

40. The system of claim 34, wherein the metal carbonate is part of a material containing metal carbonate.

41. The system of claim 40, wherein the material containing metal carbonate is a natural material, synthesized material, or waste material.

42. The system of claim 34, wherein other system is an agricultural system or aquaculture system.

43. The system of claim 34, wherein the other system is configured to apply or use the produced CO₂ as an inert gas for chemical processes, for welding, as a lasing medium, for preventing spoilage of foods and/or other air-sensitive materials, to extinguish fires, to dilute flammable and/or toxic vapours, as a non-reactive cooling gas, as a toxic gas to terminate and/or subdue animals, as a medical gas, as a propellant, as a food additive, as a reagent, as a component for the production of building materials, as a pest control mechanism, as an algae growth promotor, as a coral growth promotor, as an oil recovery pressurizing and/or flow agent, as a cleaning agent, as a solvent, and/or as a refrigerant.

44. A method, comprising:
electrochemically dissolving a metal carbonate to release CO₂; and
using the released CO₂ to improve a health, a growth rate, and/or a yield of an organism.

45. The method of claim 44, wherein electrochemically dissolving the metal carbonate to release CO₂ comprises electrochemically generating an acid to dissolve the metal carbonate.

46. The method of claim 45, wherein the metal carbonate is dissolved under a pressure greater than one atmosphere.

47. The method of claim 45, wherein electrochemically generating the acid comprises electrolyzing water to generate the acid which comprises hydrogen ions.

48. The method of claim 45, wherein the hydrogen ions react with the metal carbonate to generate metal ions and the released CO₂.

49. The method of claim 48, further comprising electrochemically generating hydroxide ions and reacting the hydroxide ions with the metal ions to form a metal hydroxide solid.

50. The method of claim 49, wherein the metal carbonate comprises calcium carbonate and the metal hydroxide solid comprises calcium hydroxide solid.

51. A system, comprising:
an electrochemical device configured to electrochemically dissolve a metal carbonate to release CO₂; and
a collection device configured to collect the released CO₂ and provide the collected CO₂ to a storage system and/or operating system, wherein the storage system and/or operating system are configured to enable the collected CO₂ to be applied to, or used by, another system for one or more purposes.

52. The system of claim 51, wherein the electrochemical device comprises an electrolyzer which is configured to electrochemically generate an acid to dissolve the metal carbonate located in the electrolyzer.

53. The system of claim 52, wherein the electrolyzer comprises:
a water tank;
two electrodes located in the water tank;
and a current or voltage source, and
wherein the water tank is configured to hold water and the metal carbonate.

54. The system of claim 53, wherein electrochemical device comprises a pressure vessel enclosing the metal carbonate located in the electrolyzer and configured such that a pressure under which the metal carbonate is dissolved is greater within the pressure vessel than a pressure outside the pressure vessel.

55. A method, comprising:
dissolving a metal carbonate using an acid to release CO₂; and
using the released CO₂ to improve a health, a growth rate, and/or a yield of an organism.
